(19) United States
(12) Patent Application Publication
(10) Pub. No.: US 2022/0062310 A1
(43) Pub. Date: Mar. 3, 2022

(54) PSILOCYBIN IN PATIENTS WITH OBSESSIVE-COMPULSIVE DISORDER

(71) Applicant: YALE UNIVERSITY, New Haven, CT (US)

(72) Inventors: Benjamin Kelmendi, New Haven, CT (US); Christopher Pittenger, Bethany, CT (US)

(21) Appl. No.: 17/466,111

(22) Filed: Sep. 3, 2021

Related U.S. Application Data
(60) Provisional application No. 63/074,275, filed on Sep. 3, 2020.

(51) Int. Cl. A61K 31/675; A61P 25/18

(52) U.S. Cl. CPC A61K 31/675; A61P 25/18

(57) ABSTRACT

This disclosure provides methods of treating or, reversing, and/or eliminating obsessive-compulsive disorder (OCD) in a subject by administering to the subject a dose of a 5-HT receptor agonist such as psilocybin. The method can significantly reduce or eliminate the symptoms of OCD with a single dose of a 5-HT receptor agonist, an effect that can last at least 12 weeks.

### CROSS-REFERENCE TO RELATED APPLICATION

This application claims priority to U.S. Provisional Patent Application Ser. No. 63/074,275 entitled “PSILOCYBIN IN PATIENTS WITH OBSESSIVE-COMPULSIVE DISORDER,” filed Sep. 3, 2020, the disclosure of which is incorporated herein by reference in its entirety.

### BACKGROUND OF THE INVENTION

OCD sufferers typically fall into several categories. Washers are obsessed with contamination and have hand washing and cleaning compulsions. Checkers are obsessed with checking things associated with harm or danger (locks, turning off the oven). Doubters are obsessed with having everything perfect or else something terrible will happen or they will be punished. Counters and arrangers are obsessed with order and symmetry (numbers, colors, arrangements). Hoarders are obsessed with keeping items and fear that something bad will happen if they throw anything away. OCD can be difficult to diagnose because these symptoms can overlap with those of depression, schizophrenia, or other disorders.

OCD affects individuals of all ages, from preschool to adulthood, and is partly genetic. There is a suggestion from research that OCD is caused by problems in communication between the frontal brain and deeper brain structures and is related to dysregulation of the neurotransmitter serotonin.

OCD is usually treated with cognitive behavior therapy and/or medication. Exposure and Response Prevention is one therapy used wherein the individual is gradually exposed to the thoughts, images, objects, and situations that make them anxious in the presence of a therapist, and the individual makes a choice not to perform a compulsive behavior when the anxiety has been triggered. Over time, this leads to a drop in the individual's anxiety level. Drawbacks to this therapy is that access to appropriately trained therapists is limited, and effective therapy requires a degree of insight and effort, and an ability to tolerate symptom discomfort that is difficult for many patients to muster. OCD is also one of the few psychiatric disorders in which invasive brain surgery is an accepted therapeutic option, which is a testament to the profound suffering it can produce.

Serotonin reuptake inhibitors (SRIs) can be used to treat OCD. Such drugs are usually administered in high doses, such as fluvoxamine (up to 300 mg/day), fluoxetine (40-80 mg/day), sertraline (up to 200 mg/day), paroxetine (40-60 mg/day), citalopram (up to 40 mg/day), clomipramine (up to 250 mg/day), escitalopram (up to 40 mg/day), and venlafaxine (up to 375 mg/day). Drawbacks with current medications include difficulty finding an effective medication or combination of medications, a long lag time (3 to 8 weeks) before an improvement of symptoms, and/or risk of suicidal thoughts or behavior. 30-40% of patients do not respond at all to SRI treatment, and most patients who do respond continue to experience problematic residual symptoms.

The neurobiological mechanisms underlying OCD remain poorly understood. More effective and faster-acting interventions are urgently needed.

### SUMMARY OF THE INVENTION

In one aspect, a method of treating, preventing, and/or ameliorating obsessive-compulsive disorder (OCD) in a subject is provided. The method includes:

administering a therapeutically effective amount of a composition comprising a 5-HT receptor agonist to the subject, wherein the subject:
i) is suffering from OCD;
ii) is not diagnosed with obsessive-compulsive personality disorder (OCPD); and

reducing, reversing, and/or eliminating OCD symptoms in the subject. The method also includes reducing, reversing, and/or eliminating obsessive thoughts and/or compulsive behaviors in the subject. Surprisingly and advantageously, in some embodiments, administering about 0.05 to about 0.55 mg/kg of psilocybin, or a salt, solvate, analog, or functional equivalent thereof, to a subject in a single dose results in a long-term reduction, reversal, and/or elimination of OCD symptoms in the subject, including obsessive thoughts and/or compulsive behaviors.

### BRIEF DESCRIPTION OF THE FIGURES

Other advantages of the present invention are readily appreciated as the same becomes better understood by reference to the following detailed description when considered in connection with the accompanying drawings.

FIG. 1 is a schematic representation of a treatment protocol with psilocybin, according to various embodiments.

FIG. 2 is a graph showing a comparison of the acute response (over 48 hours) to treatment with psilocybin (solid lines) or placebo (niacin; dashed lines) in subjects with OCD, as measured by the YBOCS (Yale-Brown Obsessive Compulsive Scale).

FIG. 3 is a graph showing a comparison of the chronic response (over 12 weeks) to treatment with psilocybin (solid lines) or placebo (niacin; dashed lines) in subjects with OCD, as measured by the YBOCS score.

FIG. 4 is a graph showing chronic response (over 12 weeks) to treatment with psilocybin in subjects with OCD who had previously received placebo.

FIG. 5 presents two graphs showing change in depression scores in subjects with OCD treated with psilocybin (solid lines, left panel) or placebo (dashed lines, left panel), and in subjects treated with psilocybin who were previously treated with placebo (right panel).

FIG. 6 is a graph showing chronic changes in trait anxiety (left panel) and state anxiety (right panel) in subjects with OCD receiving psilocybin (solid lines) or placebo (dashed lines).

FIG. 7 is a graph showing chronic changes in trait anxiety (left panel) and state anxiety (right panel) following psilocybin in subjects with OCD who previously received placebo.

FIG. 8 is a graph showing the quality of acute psychedelic experience, as measured using the Mystical Experiences Questionnaire (MEQ), in subjects with OCD receiving psilocybin (left), placebo (middle), or psilocybin after previously receiving placebo (right).

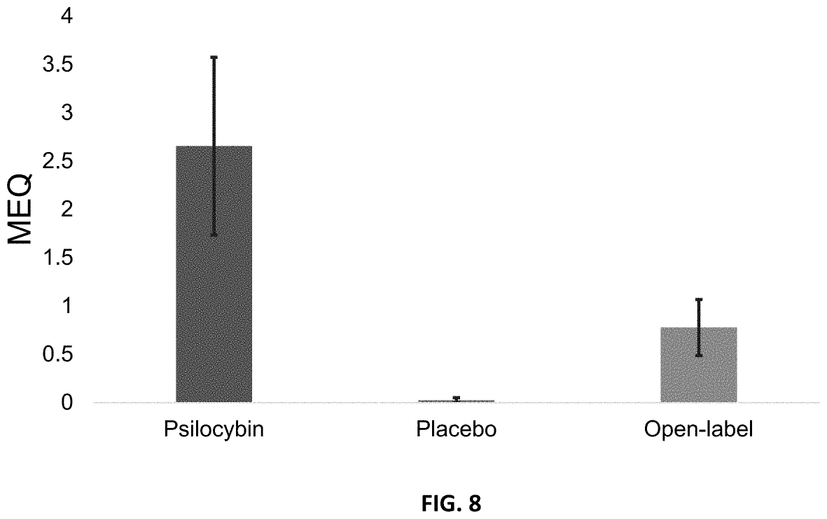

### DETAILED DESCRIPTION OF THE INVENTION

Reference will now be made in detail to certain embodiments of the disclosed subject matter. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

A “disease” is a state of health of a person or an animal wherein the person animal cannot maintain homeostasis, and wherein if the disease is not ameliorated then the person or animal's health continues to deteriorate.

In contrast, a “disorder” in a person or an animal is a state of health in which the person or animal is able to maintain homeostasis, but in which the person or animal's state of health is less favorable than it would be in the absence of the disorder. Left untreated, a disorder does not necessarily cause a further decrease in the person or animal's state of health.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

As used herein, the term “efficacy” refers to the maximal effect (Emax) achieved within an assay.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, Pa.), which is incorporated herein by reference.

The terms “patient,” “subject,” or “individual” are used interchangeably herein, and refer to any person or animal, or cells thereof whether in vitro or in situ, amenable to the methods described herein. In a non-limiting embodiment, the patient, subject or individual is a human.

As used herein, the term “potency” refers to the dose needed to produce half the maximal response (ED₅₀).

A “therapeutic” treatment is a treatment administered to a subject who exhibits signs of pathology, for the purpose of diminishing or eliminating those signs.

As used herein, the term “treatment” or “treating” is defined as the application or administration of a therapeutic agent, i.e., a compound or compounds as described herein (alone or in combination with another pharmaceutical agent), to a patient, or application or administration of a therapeutic agent to an isolated tissue or cell line from a patient (e.g., for diagnosis or ex vivo applications), who has a condition contemplated herein or a symptom of a condition contemplated herein, with the purpose to cure, heal, alleviate, relieve, alter, remedy, ameliorate, improve or affect a condition contemplated herein, or the symptoms of a condition contemplated herein. Such treatments may be specifically tailored or modified, based on knowledge obtained from the field of pharmacogenomics.

The compound of the present invention is administered and dosed in accordance with good medical practice, taking into account the clinical condition of the individual patient, the site and method of administration, scheduling of administration, patient age, sex, body weight and other factors known to medical practitioners. The pharmaceutically “effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

Methods of Treating Obsessive-Compulsive Disorder (OCD)

In certain embodiments, the present disclosure solves the problem of treating and/or ameliorating and/or preventing OCD by providing an acute dosing treatment with a 5-HT receptor agonist. In certain embodiments, such treatment results in significant and long-lasting reduction or elimination of OCD in a subject. In various embodiments, the method reduces, reverses, and/or eliminates OCD symptoms in the subject. In various embodiments, the method reduces, reverses, and/or eliminates obsessive thoughts and/or compulsive behaviors in the subject. The method also includes, in various embodiments, the step of reducing, reversing, or eliminating anxiety.

In various embodiments, a method of treating, preventing, or ameliorating obsessive-compulsive disorder (OCD) in a subject is provided. In certain embodiments, the method includes administering a therapeutically effective amount of a 5-HT receptor agonist to the subject. In certain embodiments, the subject is suffering from OCD. In certain embodiments, the subject is not diagnosed with obsessive-compulsive personality disorder (OCPD). In certain embodiments, the subject was not treated with a contraindicated medication for at least 4 weeks before starting treatment with the 5-HT receptor agonist. In certain embodiments, the method further includes reducing, reversing, and/or eliminating OCD symptoms in the subject.

The methods described herein are for treatment of OCD and not OCPD. As described in the Diagnostic and Statistical Manual of Mental Disorders, Fifth Edition [DSM-5], patients diagnosed with OCPD have a persistent pattern of preoccupation with order; perfectionism; and control of self, others, and situations.

This pattern is shown by the presence of ≥4 of the following:
Preoccupation with details, rules, schedules, organization, and lists
A striving to do something perfectly that interferes with completion of the task
Excessive devotion to work and productivity (not due to financial necessity), resulting in neglect of leisure activities and friends
Excessive conscientiousness, fastidiousness, and inflexibility regarding ethical and moral issues and values
Unwillingness to throw out worn-out or worthless objects, even those with no sentimental value
Reluctance to delegate or work with other people unless those people agree to do things exactly as the patients want
A miserly approach to spending for themselves and others because they see money as something to be saved for future disasters
Rigidity and stubbornness

OCPD is clinically distinguishable from OCD, at least because patients with OCD have true obsessions (repetitive, unwanted, intrusive thoughts that cause marked anxiety) and compulsions (ritualistic behaviors that they feel they must do to reduce their anxiety-related obsessions). Patients with OCD are often distressed by their lack of control over compulsive drives. In contrast, patients with obsessive-compulsive personality disorder, the need for control is driven by their preoccupation with order so their behavior, values, and feelings are acceptable and consistent with their sense of self.

In treating OCD, the method can include the steps of reducing, reversing, and/or eliminating obsessive thoughts and/or compulsive behaviors. For example, by administering the 5-HT receptor agonist, obsessive thoughts such as repeated thoughts about contamination, fear of losing control, repeated doubts, a need to have things in a particular order, superstitions, aggressive or horrific impulses, and sexual or violent imagery can be reduced, reversed, and/or eliminated. Compulsive behaviors reduced, reversed, and/or eliminated such as repetitive behaviors (hand washing, cleaning, ordering or checking on things such as switches, locks, appliances, accumulating junk, checking in on loved ones to make sure they are safe), or mental acts (praying, counting, tapping, or repeating words). Generally, anxiety can also be reduced, reversed, and/or eliminated through the treatment.

In various embodiments, the 5-HT receptor agonist is psilocybin, or a salt and/or solvate thereof. Psilocybin has the following structure:

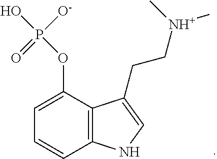 [SMILES: C[NH+](C)CCc1c[nH]c2cccc(OP(=O)([O-])O)c12]

Psilocybin (3-(2-dimethylaminoethyl)-1H-indol-4-yl] dihydrogen phosphate) is a psychedelic drug that is produced by psilocybin mushrooms, such as, but not limited to, P. azurescens, P. semilanceata, and P. cyanescens. It is converted in the body to psilocin (the active molecule) and acts as a partial agonist for serotonin 5-hydroxytryptamine (5-HT) receptors (with most affinity to 5-HT_{2A} and 5-HT_{2C}, and lesser affinity to 5-HT_{1A}). Psilocybin can also indirectly increase the concentration of dopamine in the brain. The psilocybin used in the present invention can be naturally derived or synthetic. The psilocybin used in the present invention can have a purity that is equal to or higher than about 80%, 81%, 82%, 83%, 84%, 85%, 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, and/or 100%. Homologs thereof and analogs thereof can also be used. Any functional equivalents can also be used, i.e. any compound that provides the same function as psilocybin. One example of an analog is psilacetin (O-Acetylpsilocin), which is also converted to psilocin in the body. Baeocystin is another analog ([3-(2-methylaminoethyl)-1H-indol-4-yl] dihydrogen phosphate).

Dosing

The therapeutically effective amount or dose of a compound described herein depends on the age, sex, and weight of the patient, the current medical condition of the patient and the progression of OCD in the patient being treated. The compound of the present invention is administered and dosed in accordance with good medical practice, taking into account the clinical condition of the individual patient, the site and method of administration, scheduling of administration, patient age, sex, body weight and other factors known to medical practitioners.

The doses can be single doses or multiple doses over a period of several days. The treatment generally has a length proportional to the length of the disease process and drug effectiveness and the patient species being treated.

In various embodiments, the therapeutically effective amount is a dose of psilocybin is about 0.05 to about 0.55 mg/kg, about 0.1 to about 0.4 mg/kg, or about 0.2 to about 0.3 mg/kg. In various embodiments, the dose of psilocybin is at least about, greater than, or equal to about 0.05, 0.1, 0.15, 0.2, 0.25, 0.3, 0.35, 0.4, 0.45, 0.5, or about 0.55 mg/kg. In various embodiments, the dose of psilocybin is at least about, less than, or equal to about 1, 2, 3, 4, 5, 6, 7, 8, 9, 10, 11, 12, 13, 14, 15, 6, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mg. In various embodiments, the dose of psilocybin is 0.25 mg/kg. The psilocybin, in various embodiments, is at least 98, 99, 99.5, or 99.9% pure psilocybin and does not contain any portion, residue, and/or additional compound(s) from mushrooms. The psilocybin can also be administered with one or more pharmaceutically acceptable carriers in any of the formulations or dosage forms described herein.

It was surprisingly and unexpectedly discovered that long-lasting remission or elimination of OCD symptoms and associated behaviors can be achieved by administering a single dose of psilocybin as described herein. In various embodiments, the method includes administering a single dose of 0.05 to about 0.55 mg/kg psilocybin, which results in a reduction or elimination of OCD symptoms and associated behaviors for a period of at least or equal to 1, 2, 3, 4, 5, 6, 7, 8, 9, 10, 11, or 12 weeks, or more. OCD symptoms and associated behaviors include obsessive thoughts and/or compulsive behaviors in the subject. In various embodiments, the obsessive thoughts are selected from the group consisting of repeated thoughts about contamination, fear of losing control, repeated doubts, a need to have things in a particular order, superstitions, aggressive or horrific impulses, and sexual or violent imagery. In various embodiments, the compulsive behaviors are selected from the group consisting of repetitive acts and mental acts.

In various embodiments, administering a single dose of psilocybin results in a reduction of elimination of OCD symptoms and associated behaviors for a period of at least or equal to 1 week. In various embodiments, administering a single dose of psilocybin results in a reduction or elimination of OCD symptoms and associated behaviors for a period of at least or equal to 2 weeks. In various embodiments, administering a single dose of psilocybin results in a reduction or elimination of OCD symptoms and associated behaviors for a period of at least or equal to 3 weeks. In various embodiments, administering a single dose of psilocybin results in a reduction or elimination of OCD symptoms and associated behaviors for a period of at least or equal to 4 weeks. In various embodiments, administering a single dose of psilocybin results in a reduction or elimination of OCD symptoms and associated behaviors for a period of at least or equal to 5 weeks. In various embodiments, administering a single dose of psilocybin results in a reduction or elimination of OCD symptoms and associated behaviors for a period of at least or equal to 6 weeks. In various embodiments, administering a single dose of psilocybin results in a reduction or elimination of OCD symptoms and associated behaviors for a period of at least or equal to 7 weeks. In various embodiments, administering a single dose of psilocybin results in a reduction or elimination of OCD symptoms and associated behaviors for a period of at least or equal to 8 weeks. In various embodiments, administering a single dose of psilocybin results in a reduction or elimination of OCD symptoms and associated behaviors for a period of at least or equal to 9 weeks. In various embodiments, administering a single dose of psilocybin results in a reduction or elimination of OCD symptoms and associated behaviors for a period of at least or equal to 10 weeks. In various embodiments, administering a single dose of psilocybin results in a reduction or elimination of OCD symptoms and associated behaviors for a period of at least or equal to 11 weeks. In various embodiments, administering a single dose of psilocybin results in a reduction or elimination of OCD symptoms and associated behaviors for a period of at least or equal to 12 weeks. In various embodiments, administering a single dose of 0.25 mg/kg psilocybin results in a reduction or elimination of OCD symptoms and associated behaviors for a period of at least or equal to 12 weeks. The Examples show unexpectedly that substantially pure psilocybin administered in just a single dose can provide long term effects in treating OCD.

The single dose of psilocybin can be any of the dosage amounts described herein. In various embodiments, the reduction or elimination of OCD symptoms and associated behaviors is achieved by administering more than a single dose of psilocybin, such as two, three, four, or more doses. The period between doses optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. If multiple psilocybin doses are administered, the doses after the first dose of psilocybin can be the same as the first dose or can be reduced doses. The dose reduction during subsequent doses can be from 10%-95%, including, by way of example only, 10%, 15%, 20%, 25%, 30%, 35%, 40%, 45%, 50%, 55%, 60%, 65%, 70%, 75%, 80%, 85%, 90%, or 95%.

Once improvement of the patient's conditions has occurred, a maintenance dose is administered, if necessary. Subsequently, the dosage or the frequency of administration, or both, is reduced to a level at which the improved disease is retained. In certain embodiments, patients require intermittent treatment on a long-term basis upon any recurrence of symptoms. In various embodiments, the subject is a mammal. In various embodiments, the subject is a human.

In some embodiments, the non-treatment period after administering a single dose of a 5-HT receptor agonist, such as psilocybin, is at least or equal to 1, 2, 3, 4, 5, 6, 7, 8, 9, 10, 11, or 12 weeks, or more. During the non-treatment period the 5-HT receptor agonist administered in the first dose, such as psilocybin, is not administered to the subject. If a subject's OCD symptoms begin to return, then a subsequent dose of a 5-HT receptor agonist, such as psilocybin, can be administered as described herein. This type of periodic single-dose treatment can continue for any number of months or years, or for the life-time of the subject. In some embodiments, at least one subsequent single dose administration of a 5-HT receptor agonist, such as psilocybin, occurs after the first single dose treatment of the subject. The determination of when a subsequent single dose treatment is administered, in some embodiments, is based on an increase in the subject's YBOCS score over time. The magnitude of the increase that warrants subsequent treatment can be determined by a physician of skill in the art. In some embodiments, the increase in the subject's YBOCS score that warrants a subsequent treatment is at least or equal to 2, 3, 4, 5, 6, 7, 8, 9, or 10 as compared to the subject's prior average, maximum, or minimum YBOCS score for the preceding 1, 2, 3, 4, 5, 6, days, or the preceding 1, 2, 3, 4, 5, 6, 7, 8, 9, 10, 11, or 12 weeks, or any portion thereof such as 1.5 weeks, and the like.

Measurement of Reduction of OCD Symptoms/Behaviors

In various embodiments, the reduction, reversal, and/or elimination of OCD symptoms in the subject is measured by the Yale-Brown Obsessive Compulsive Scale (YBOCS). In various embodiments, the subject has a pre-treatment YBOCS score of at least about 18, 19, or 20. A pre-treatment YBOCS score is the individual's score prior to administration of any 5-HT receptor agonist, such as psilocybin. In various embodiments, subjects with YBOCS scores below 18 are not suitable candidates for treatment with the methods described herein. In various embodiments, one week after administering the psilocybin, the subject has a reduced YBOCS score that is at least about 25-40% smaller than the pre-treatment YBOCS score. In various embodiments, one week after administering the psilocybin, the subject has a reduced YBOCS score that is at least about 25, 26, 27, 28, 29, 30, 31, 32, 33, 34, 35, 36, 37, 38, 39, or 40% smaller than the pre-treatment YBOCS score. In various embodiments, at least one week after administering the 5-HT receptor agonist, the subject has a YBOCS score that is at least about 25% smaller than the pre-administration YBOCS score.

In various embodiments, the reduced YBOCS score persists for at least about 2, 3, 4, 5, 6, 7, 8, 9, 10, 11, or 12 weeks. In various embodiments, the reduced YBOCS score persists for at least about 8 weeks after administration of the 5-HT receptor agonist.

Medical Contraindications for Treatment

In various embodiments, contraindicated medications include anti-seizure medications, insulin and oral hypoglycemics, certain hypertension medications (clonidine and aldomet specifically), cardiovascular medications and the following psychotropic medications: anti-psychotics (first and second generation agents), anti-depressants, and mood stabilizers.

Examples of oral hypoglycemics include sulfonylureas such as Acetohexamide (Dymelor), Chlorpropamide (Diabinese), Tolazamide (Tolinase), Tolbutamide (Orinase), Glyburide (Micronase), Glipizide (Glucotrol), Glimepiride (Amaryl), and the like; meglitinides such as Repaglinide (Prandin), Nateglinide (Starlix), and the like; biguanides such as Metformin (Glucophage); thiazolidinediones such as Pioglitazone (Actos), Rosiglitazone (Avandia), and the like; and Alpha-glucosidase inhibitors such as Acarbose (Precose), Miglitol (Glycet), and the like.

Examples of cardiovascular medications include anticoagulants such as Apixaban (Eliquis), Dabigatran (Pradaxa), Edoxaban (Savaysa), Heparin (various), Rivaroxaban (Xarelto), Warfarin (Coumadin), and the like; antiplatelet agents such as aspirin, Clopidogrel (Plavix), Dipyridamole (Persantine), Prasugrel (Effient), Ticagrelor (Brilinta); ACE inhibitors such as Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), Trandolapril (Mavik), and the like; angiotensin II receptor inhibitors such as Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), Trandolapril (Mavik), and the like; beta blockers such as Acebutolol (Sectral), Atenolol (Tenormin), Betaxolol (Kerlone), Bisoprolol/hydrochlorothiazide (Ziac), Bisoprolol (Zebeta), Metoprolol (Lopressor, Toprol XL), Nadolol (Corgard), Propranolol (Inderal), Sotalol (Betapace) and the like; calcium channel blockers such as Amlodipine (Norvasc), Diltiazem (Cardizem, Tiazac), Felodipine (Plendil), Nifedipine (Adalat, Procardia), Nimodipine (Nimotop), Nisoldipine (Sular), Verapamil (Calan, Verelan) and the like; statins such as Atorvastatin (Lipitor), Fluvastatin (Lescol), Lovastatin (Mevacor), Pitavastatin (Livalo), Pravastatin (Pravachol), Rosuvastatin (Crestor), Simvastatin (Zocor) and the like; digitalis preparations such as dioxin; diuretics such as Acetazolamide (Diamox), Amiloride (Midamor), Bumetanide (Bumex), Chlorothiazide (Diuril), Chlorthalidone (Hygroton), Furosemide (Lasix), Hydro-chlorothiazide (Esidrix, Hydrodiuril), Indapamide (Lozol), Metalozone (Zaroxolyn), Spironolactone (Aldactone), Torsemide (Demadex) and the like; and vasodialators such as Isosorbide dinitrate (Isordil), Isosorbide mononitrate (Imdur), Hydralazine (Apresoline), Nitroglycerin (Nitro Bid, Nitro Stat), and Minoxidil.

Psychotropic medications include antipsychotics such as Thorazine (chlorpromazine), Trilafon (perphenazine), Stelazine (trifluoperazine), Serentil (mesoridazine), Prolixin (fluphenazine), Navane (thiothixene), Moban (molindone), Mellaril (thioridazine), Loxitane (loxapine), Haldol (haloperidol), and the like; atypical antipsychotics such as Abilify (aripiprazole), Clozaril (clozapine), Geodon (ziprasidone), Risperdal (risperidone), Seroquel (quetiapine), Zyprexa (olanzapine), and the like; antidepressants such as Celexa (citalopram), Lexapro (escitalopram), Luvox (fluvoxamine), Paxil (paroxetine), Prozac (fluoxetine), Zoloft (sertraline); monoamine oxidase inhibitors (MAOIs) such as Emsam (selegiline), Marplan (isocarboxazid), Nardil (phenelzine), Parnate (tranylcypromine), and the like; tricyclics (TCAs) such as Anafranil (clomipramine), Asendin (amoxapine), Elavil (amitriptyline), Norpramin (desipramine), Pamelor (nortriptyline), Sinequan (doxepin), Surmontil (trimipramine), Tofranil (imipramine), Vivactil (protiptyline), and the like; serotonin norepinephrine reuptake inhibitors (SNRIs) such as Pristiq (desvenlafaxine), Effexor (venlafaxine), Cymbalta (duloxetine), and the like; anti-anxiety/anti-panic medications such as Ativan (lorazepam), BuSpar (buspirone), Inderal (propranolol), Klonopin (clonazepam), Librium (chlordiazepoxide), Serax (oxazepam), Tenormin (atenolol), Tranxene (clorazepate), Valium (diazepam), Xanax (alprazolam), and the like; stimulants such as Adderall (amphetamine and dextroamphetamine), Dexedrine (dextroamphetamine), Ritalin (methylphenidate), and the like; and mood stabilizers such as Lamictal (lamotrigine), lithium, and the like.

In various embodiments, the subject cannot have taken any anti-seizure medications, insulin, oral hypoglycemics, certain hypertension medications, cardiovascular medications, and/or psychotropic medications described herein within 1, 2, 3, 4, 5, 6, 7, 8, 9, 10, 11, or 12 weeks prior to commencing therapy according to the methods described herein. In various embodiments, the preceding medications cannot have been taken within 2 weeks of commencing therapy as described herein. In various embodiments, the preceding medications cannot have been taken within 3 weeks of commencing therapy as described herein. In various embodiments, the preceding medications cannot have been taken within 4 weeks of commencing therapy as described herein. In various embodiments, the preceding medications cannot have been taken within 5 weeks of commencing therapy as described herein. In various embodiments, the preceding medications cannot have been taken within 6 weeks of commencing therapy as described herein. In various embodiments, the preceding medications cannot have been taken within 7 weeks of commencing therapy as described herein. In various embodiments, the preceding medications cannot have been taken within 8 weeks of commencing therapy as described herein. In various embodiments, the preceding medications cannot have been taken within 9 weeks of commencing therapy as described herein. In various embodiments, the preceding medications cannot have been taken within 10 weeks of commencing therapy as described herein. In various embodiments, the preceding medications cannot have been taken within 11 weeks of commencing therapy as described herein. In various embodiments, the preceding medications cannot have been taken within 12 weeks of commencing therapy as described herein.

In various embodiments, the subject has a YBOCS score of at least 19 and has not taken one or more of any of the contraindicated medications as described herein for a period of at least 2, 3, 4, 6, 7, 8, 9, 11, or 12 weeks.

Formulations and Dosage Forms

The compounds described herein can be formulated in a pharmaceutical composition comprising at least one compound as described herein and at least one pharmaceutically acceptable carrier. In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration. It should be noted that the psilocybin can be administered as the compound and can be administered alone or as an active ingredient in combination with pharmaceutically acceptable carriers, diluents, adjuvants and vehicles.

In various embodiments, the psilocybin can be administered orally, subcutaneously or parenterally including intravenous, intraarterial, intramuscular, intraperitoneally, intratonsillar, and intranasal administration as well as intrathecal and infusion techniques. Implants of the compounds are also useful. The patient being treated is a warm-blooded animal and, in particular, mammals including humans. The pharmaceutically acceptable carriers, diluents, adjuvants and vehicles as well as implant carriers generally refer to inert, non-toxic solid or liquid fillers, diluents or encapsulating material not reacting with the active ingredients such as psilocybin. In various embodiments, the 5-HT receptor agonist, such as psilocybin, is administered orally.

The doses can be single doses or multiple doses over a period of several days. The treatment generally has a length proportional to the length of the disease process and drug effectiveness and the patient species being treated.

When administering the compound of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Compositions as described herein can be prepared, packaged, or sold in a formulation suitable for oral or buccal administration. A tablet that includes a compound as described herein can, for example, be made by compressing or molding the active ingredient, optionally with one or more additional ingredients. Compressed tablets may be prepared by compressing, in a suitable device, the active ingredient in a free-flowing form such as a powder or granular preparation, optionally mixed with one or more of a binder, a lubricant, an excipient, a surface active agent, and a dispersing agent. Molded tablets may be made by molding, in a suitable device, a mixture of the active ingredient, a pharmaceutically acceptable carrier, and at least sufficient liquid to moisten the mixture. Pharmaceutically acceptable excipients used in the manufacture of tablets include, but are not limited to, inert diluents, granulating and disintegrating agents, dispersing agents, surface-active agents, disintegrating agents, binding agents, and lubricating agents.

Suitable dispersing agents include, but are not limited to, potato starch, sodium starch glycollate, poloxamer 407, or poloxamer 188. One or more dispersing agents can each be individually present in the composition in an amount of about 0.01% w/w to about 90% w/w relative to weight of the dosage form. One or more dispersing agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Surface-active agents (surfactants) include cationic, anionic, or non-ionic surfactants, or combinations thereof. Suitable surfactants include, but are not limited to, behentrimonium chloride, benzalkonium chloride, benzethonium chloride, benzododecinium bromide, carbethopendecinium bromide, cetalkonium chloride, cetrimonium bromide, cetrimonium chloride, cetylpyridine chloride, didecyldimethylammonium chloride, dimethyldioctadecylammonium bromide, dimethyldioctadecylammonium chloride, domiphen bromide, lauryl methyl gluceth-10 hydroxypropyl dimonium chloride, tetramethylammonium hydroxide, thonzonium bromide, stearalkonium chloride, octenidine dihydrochloride, olaflur, N-oleyl-1,3-propanediamine, 2-acrylamido-2-methylpropane sulfonic acid, alkylbenzene sulfonates, ammonium lauryl sulfate, ammonium perfluorononanoate, docusate, disodium cocoamphodiacetate, magnesium laureth sulfate, perfluorobutanesulfonic acid, perfluorononanoic acid, perfluorooctanesulfonic acid, perfluorooctanoic acid, potassium lauryl sulfate, sodium alkyl sulfate, sodium dodecyl sulfate, sodium laurate, sodium laureth sulfate, sodium lauroyl sarcosinate, sodium myreth sulfate, sodium nonanoyloxybenzenesulfonate, sodium pareth sulfate, sodium stearate, sodium sulfosuccinate esters, cetomacrogol 1000, cetostearyl alcohol, cetyl alcohol, cocamide diethanolamine, cocamide monoethanolamine, decyl glucoside, decyl polyglucose, glycerol monostearate, octylphenoxypolyethoxyethanol CA-630, isoceteth-20, lauryl glucoside, octylphenoxypolyethoxyethanol P-40, Nonoxynol-9, Nonoxynols, nonyl phenoxypolyethoxylethanol (NP-40), octaethylene glycol monododecyl ether, N-octyl beta-D-thioglucopyranoside, octyl glucoside, oleyl alcohol, PEG-10 sunflower glycerides, pentaethylene glycol monododecyl ether, polidocanol, poloxamer, poloxamer 407, polyethoxylated tallow amine, polyglycerol polyricinoleate, polysorbate, polysorbate 20, polysorbate 80, sorbitan, sorbitan monolaurate, sorbitan monostearate, sorbitan tristearate, stearyl alcohol, surfactin, Triton X-100, and Tween 80. One or more surfactants can each be individually present in the composition in an amount of about 0.01% w/w to about 90% w/w relative to weight of the dosage form. One or more surfactants can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable diluents include, but are not limited to, calcium carbonate, magnesium carbonate, magnesium oxide, sodium carbonate, lactose, microcrystalline cellulose, calcium phosphate, calcium hydrogen phosphate, and sodium phosphate, Cellactose® 80 (75% □-lactose monohydrate and 25% cellulose powder), mannitol, pre-gelatinized starch, starch, sucrose, sodium chloride, talc, anhydrous lactose, and granulated lactose. One or more diluents can each be individually present in the composition in an amount of about 0.01% w/w to about 90% w/w relative to weight of the dosage form. One or more diluents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable granulating and disintegrating agents include, but are not limited to, sucrose, copovidone, corn starch, microcrystalline cellulose, methyl cellulose, sodium starch glycollate, pregelatinized starch, povidone, sodium carboxy methyl cellulose, sodium alginate, citric acid, croscarmellose sodium, cellulose, carboxymethylcellulose calcium, colloidal silicone dioxide, crosspovidone and alginic acid. One or more granulating or disintegrating agents can each be individually present in the composition in an amount of about 0.01% w/w to about 90% w/w relative to weight of the dosage form. One or more granulating or disintegrating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable binding agents include, but are not limited to, gelatin, acacia, pre-gelatinized maize starch, polyvinylpyrrolidone, anhydrous lactose, lactose monohydrate, hydroxypropyl methylcellulose, methylcellulose, povidone, polyacrylamides, sucrose, dextrose, maltose, gelatin, polyethylene glycol. One or more binding agents can each be individually present in the composition in an amount of about 0.01% w/w to about 90% w/w relative to weight of the dosage form. One or more binding agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Suitable lubricating agents include, but are not limited to, magnesium stearate, calcium stearate, hydrogenated castor oil, glyceryl monostearate, glyceryl behenate, mineral oil, polyethylene glycol, poloxamer 407, poloxamer 188, sodium laureth sulfate, sodium benzoate, stearic acid, sodium stearyl fumarate, silica, and talc. One or more lubricating agents can each be individually present in the composition in an amount of about 0.01% w/w to about 90% w/w relative to weight of the dosage form. One or more lubricating agents can each be individually present in the composition in an amount of at least, greater than, or less than about 0.01%, 0.05%, 0.1%, 0.5%, 1%, 2%, 3%, 4%, 5%, 10%, 15%, 20%, 25%, 30%, 35%, 40%, 45%, 50%, 55%, 60%, 65%, 70%, 75%, 80%, 85%, or 90% w/w relative to weight of the dosage form.

Tablets can be non-coated or they may be coated using known methods to achieve delayed disintegration in the gastrointestinal tract of a subject, thereby providing sustained release and absorption of the active ingredient. By way of example, a material such as glyceryl monostearate or glyceryl distearate may be used to coat tablets. Further by way of example, tablets may be coated using methods described in U.S. Pat. Nos. 4,256,108; 4,160,452; and 4,265,874 to form osmotically controlled release tablets. Tablets may further comprise a sweetening agent, a flavoring agent, a coloring agent, a preservative, or some combination of these in order to provide for pharmaceutically elegant and palatable preparation.

Tablets can also be enterically coated such that the coating begins to dissolve at a certain pH, such as at about pH 5.0 to about pH 7.5, thereby releasing a compound as described herein. The coating can contain, for example, EUDRAGIT® L, S, FS, and/or E polymers with acidic or alkaline groups to allow release of a compound as described herein in a particular location, including in any desired section(s) of the intestine. The coating can also contain, for example, EUDRAGIT® RL and/or RS polymers with cationic or neutral groups to allow for time controlled release of a compound as described herein by pH-independent swelling.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol. Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as such as lauryl, stearyl, or oleyl alcohols, or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets, that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In one embodiment, the compound(s) described herein are administered to a patient, alone or in combination with another pharmaceutical agent, using a sustained release formulation. In one embodiment, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### EXAMPLES

Various embodiments of the present application can be better understood by reference to the following Examples which are offered by way of illustration. The scope of the present application is not limited to the Examples given herein.

### Example 1

This study investigates the effects of oral psilocybin on OCD symptomatology and provides the first evidence of the neural mechanism that may mediate psilocybin's purported therapeutic effects on OCD, using a randomized active-placebo-controlled, double-blind study design, coupled with state-of-the-art functional neuroimaging.

### Research Methods and Design

This study pilots a single-center, randomized, active-placebo-controlled, double-blind design to examine the clinical and neural effects on OCD, of either 0.25 mg/kg of psilocybin or active placebo-control agent (niacin 250 mg), given along with non-drug preparatory and follow-up support appointments to 30 study participants.

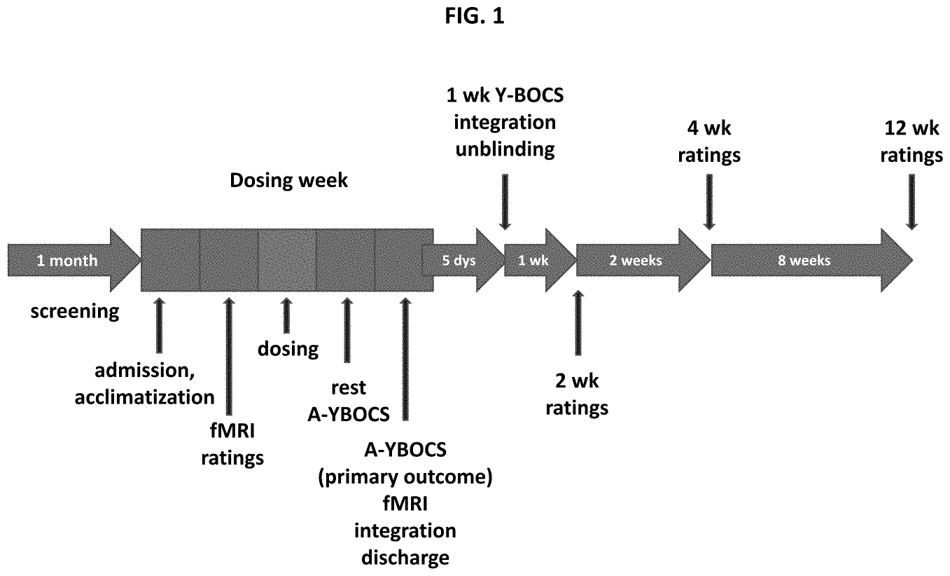

The duration of the randomized study phase is from consent until two weeks after drug administration. Participants can be followed for 12 weeks (approximately 3 months) post-study drug administration. A schematic of the research study is depicted in FIG. 1.

Patient Population—Subjects with OCD: Participants meeting study inclusion/exclusion criteria after careful screening and examination can be invited to participate and provided with all the information needed to provide an informed consent. 30 adult participants (21 to 65 years) of both sexes can be recruited, with clinically significant obsession and/or compulsions, evidenced by a YBOCS score of >18.

Eligible participants can be admitted to the CNRU at CMHC as an inpatient for at least 3 nights/4 days (visits 3, 4, 5, 6) surrounding the initial drug administration (or more, at the option of the subject and the investigator). Participants can be randomized into active medication and active-placebo-control groups and will be blinded as to their study condition. This admission 2 nights prior to the drug administration allows the participant to adjust to sleeping on the unit and allows them to settle into the research unit routine. A return (visit 7) for an fmri scan (48 hours after the administration session, v 5) is scheduled. The blind can be broken at 48 hours (visit 7), and the participants who received active-placebo-control can be offered the option to receive open-label psilocybin (as soon as two-weeks after the initial study administration).

In addition to the baseline and follow-up assessments of the participants' mood, anxiety, quality of life and spiritual beliefs and medical evaluation, the study comprises at least two introductory psychoeducation sessions with the PI and/or study personnel, study debriefing and follow-up support appointments 24 and 48 hours (v6, v7), 1 week (v8) and 2 weeks (v9) after drug administration. Participants also undergo two different fMRI sessions: 24 hours prior (v4) to drug administration and 48 hours' post-drug (v7). Participants are asked to designate 2 to 3 adults (spouse or other family members, friends, or co-workers) who can observe changes in the participants' behavior and attitude and report as part of the follow-up. The battery of follow-up assessments and measures can be repeated 1 week, 2 weeks, 4 weeks, 8 weeks, and 12 weeks post drug administration.

The Open label phase for participants who were assigned to the active-placebo control condition can follow similar procedure to the randomized phase.

Pre-Study Methods/Clinic Phone Screening—all Subjects:

Prior to participation in this research, all subjects can receive an abbreviated Qualtrics (or REDCap) screening survey and then if still eligible, can be screened by phone to determine preliminary eligibility. All subjects are informed about the Yale OCD Research Clinic, the phone screening, what the screening process involves, and the current research studies. All potential participants are told that demographic information, relevant medical history, and psychiatric history are collected during the screening process. All patients are informed that participation involves the creation of a medical chart that will identify him/her with CMHC, include registration blood draws and urine tests, and that he/she has the choice whether or not to participate or to have their information taken down. All willing adults complete this phone screen which is reviewed by the clinic to ascertain preliminary eligibility. This phone screen can be entered directly into REDCap and is considered a source document.

All seemingly eligible adult subjects are asked to come in for baseline screenings on the CNRU in the Yale OCD Research Clinic. As part of this evaluation and CMHC registration process, each subject can be told that the visit includes standard laboratory admission tests for which the medical staff collects blood and urine and reminded of the CMHC medical chart creation. All participants must give permission for the investigators to review medical records, to undergo a psychiatric evaluation and to complete a urinary drug screen to ensure their eligibility. Subjects are free to decline participation in the clinic at CMHC and to withdraw from consideration.

Clinic Screening and Patient Registration (Standard Clinic Operations):

All potential research participants who agree to be screened and registered as CMHC/clinic patients and interviewed by the clinic staff and can have a CMHC medical chart created for them (paper copy and/or online). All agreeable patients have blood drawn and urine specimens taken for CMHC clinical laboratory tests (including chemistries, hematological, thyroid function tests, pregnancy tests when applicable, urine drug screen and routine urinalysis). If the urine drug screen or the pregnancy tests are positive (except in the case of prescribed benzodiazepines which are allowed up to three days before the session), then the subject is not able to participate in the study. Laboratory results become part of that permanent CMHC medical chart and may later be included in a research case file for this study.

All seemingly eligible participants have a clinical diagnostic interview with a trained clinician, including the Mini International Neuropsychiatric Interview (MINI), the Yale-Brown Obsessive-Compulsive Symptom Checklist, the Yale-Brown Obsessive-Compulsive Scale (Y-BOCS); the Brown Assessment of Beliefs Scale (BABS) to assess insight, and the Yale Global Tic Severity Scale (YGTSS-Clinician) to assess current and past tics, and CGI-Clinician to assess overall clinical status. At this baseline clinic screening, information/data is collected utilizing standard screening instruments, both self-reports and clinician-administered reports (either in paper-format or utilizing secure web-based data capture applications, e.g., REDCap or Qualtrics) and also includes: demographics like gender, age, racial and ethnic background, BDI, MADRS, CGI-Self-Report, and the Sheehan Disability Scale. These clinic ratings aid in establishing whether a client meets initial criteria for study participation.

All subjects are asked to complete a MR Safety Questionnaire that is used by the Magnetic Resonance Research Center (MRRC) at Yale to determine the presence of any implanted metallic objects that may exclude a subject from being safely scanned or to indicate areas that may require further investigation, (e.g. pre-MRI x-ray to rule out metal in orbits).

To expand the clinic knowledge base of what underlies OCD, participants are given baseline research measures of obsessive-compulsive dimensions (symptoms, life attitudes, motivation, ideation, core beliefs) and co-occurring conditions as self-reports—Life Orientation Test Revised (LOT-R), Magical Ideation Scale (MIS), Obsessive Beliefs Questionnaire (OBQ-44), OC Trait Core Dimensions Questionnaire-Incompleteness-Harm-Avoidance (OC-TCDQ-R2), Padua Inventory of Obsessive Compulsive Disorder Symptoms (Padua), Quality of Life and Satisfaction Questionnaire Short Form (QOLESQ-SF), Responsibility Attitude Scale (RAS), Thought-Action-Fusion Scale (TAF) and general demographics questions; and as clinician administered—Cognitive Difficulties Scale, and Saving Inventory-Revised. Other measures to assess co-occurring conditions (stress and anxiety disorders, tics, trichotillomania, or hoarding) can be used as needed. These clinic measures aid in establishing whether a client meets initial criteria for study participation. Some of these standard clinic measures are repeated in the study ratings battery at the final follow-up assessment.

All preliminary clinic screening information are discussed and reviewed by the principal investigator and the entire research team. Participants may give permission for the research investigators/staff to review the participants' medical records, which is decided on a case-by-case basis upon the discretion of the PI. All adult patients who are still considered eligible based on the study inclusion and exclusion criteria and review of the baseline information from this Yale OCD Research Clinic pool, are asked to consider the consent for this study.

As the clinic has in previous studies, all patients who provide an informed consent and who agree to participant in the study are assessed. All participants must give permission for the research investigators/staff to review medical records and sign a medical release for the researchers to communicate with the participant's current provider about their treatment and participation in the study. This study uses primarily non-invasive measures of neuropsychological assessment, cognitive testing, magnetic resonance brain structure and function scans (at baseline and after treatment with the study agent), and a series of follow-up assessments over about 3 months. Also, genetic specimens can be obtained from all subjects to be placed in a repository and used to investigate the genetics of OCD, and the genetic predictors of treatment response. Subjects with OCD can be compared with active-placebo-controls.

Screening for Out of State Participants

Some potential participants live in another state and it may not feasible to have them come to the CMHC for an in-person screening. In this case the screening can be conducted utilizing secure video communication platforms (i.e. Zoom, Skype, etc.). These participants are asked to send their last physical, EKG, and blood work, that was done by their PCP. This can be faxed to the Kelmendi Clinic and reviewed by the principal investigator and the research team. A physical, EKG, and blood draw can be performed when they arrive for the inpatient stay.

Research Materials and Measures: Participants can complete self-report and clinician-administered measures of OCD symptoms, anxiety, depression, quality of life, and spirituality at baseline, prior to psilocybin session, and during all designated study visits. All subjects are asked to complete an extended set of research assessments: to examine symptom dimensions, life attitudes, motivation, ideation, core beliefs, and co-occurring conditions.
Patient Screening Visit:
Demographics (15 minutes)
Mill Safety Questionnaire (5 minutes)
Mini International Neuropsychological Interview 7.0 (MINI-7) (30-50 minutes)
Yale-Brown Obsessive-Compulsive Scale (YBOCS)
Y-BOCS Symptom Checklist (15-20 minutes)
SCID-2 Personality Questionnaire (SCID-II/PQ) (10-20 minutes)
Clinician Administered Ratings:
Montgomery-Asberg Depression Rating Scale (MADRS) (6-8 minutes)
Brown Assessment of Beliefs Scale (BABS) (4-6 minutes)
Columbia Suicide Severity Rating Scale (C-SSRS) (5 minutes)
Acute YBOCS (A-YBOCS)-specific participant OCD symptoms over prior 24 hours (5 minutes)
Y-BOCS—overall obsessive-compulsive symptoms over prior week (5-10 minutes)
Clinical Global Impression-Clinician (CGI-C) (2 min)
Yale Global Tic Severity Scale (YGTSS-Clinician) (3-10 minutes)
Community Observer Rating of Changes in Subjects' Behavior and Attitudes (COM-R) (5-10 minutes)
Self-Report Ratings:
Becks Depression Inventory (BDI) (5 minutes)
Obsessive Beliefs Questionnaire-44 (OBQ-44) (5-10 minutes)
Obsessive-Compulsive Inventory-Revised (OCI-R) (5 minutes)
Obsessive-Compulsive Trait Core Dimensions Questionnaire (OC-TCDQ) (3-5 minutes)
Thought Action Fusion Scale (TAF) (3-5 minutes)
Responsibility Attitude Scale (RAS) (5 minutes)
State-Trait Anxiety Inventory (STAI) (5 minutes)
Quality of Life Enjoyment & Satisfaction Questionnaire (Q-LESQ-SF) (3 minutes)
Mystical Experience Questionnaire (MEQ) (5 minutes)
Post-Session Subjective Experience Questionnaire (PSEQ) (3 minutes)
Reactions to Research Participation Questionnaire (RRPQ) (3-5 minutes)
Big Five Aspect Scales (BFAS) (10 minutes)
Schedule for Meaning in Life Evaluation (SMiLE) (10 minutes)
Challenging Experience Questionnaire (CEQ) (5 minutes)
5-Dimension-Altered States of Consciousness (5D-ASC) (10-15 minutes)
Positive and Negative Affect Schedule Expanded Form (PANAS-X) (10-15 minutes)
Persisting Effects Questionnaire (PEQ) (10 minutes)
Nature Relatedness Scale (NRS) (3 minutes)
Pro-Environmental Behavior Scale (PEBS) (3 minutes)
Individual Differences in Anthropomorphism Questionnaire (IDAQ) (3 minutes)
Mind-Body Dualism Scale (MBDS) (3 minutes)
Inclusion of Others in Self Scale (IOS) (1 minute)
Ethical Positions Questionnaire (EPQ) (3 minutes)
Alcohol Use Disorders Identification Test (AUDIT) (3 minutes)
Utilization of Facility and Emergent Care (UFEC) (3 minutes)
Drug Use Disorders Identification Test (DUDIT) (3 minutes)
Self-reported Nicotine Use (SRNU) (3 minutes)
Pittsburgh Sleep Quality Index (PSQI) (3 minutes)
Visual Analogue Scale (VAS) (2 minutes)
Behavior Identification Form (BIF) (3 minutes)
Magical Ideation Scale (MIS) (3 minutes)
University of Rhode Island Change Assessment (URICA) (3 minutes)
CGI-Self Report (2 minutes)
Physical Symptoms (Saftee) Checklist (PSC) (3 minutes)
Sheehan Disability Scale (SDS) (2 minutes)
Life Orientation Test Revised (LOT-R) (2 minutes)
Padua Inventory of OCD symptoms (5 minutes)
Immunological Measures/Panel: (Random phase: visits 2 (screen), 7 (48 hours post), 9 (2 weeks post), 12 (12 weeks post); and Open Label phase: visits 12 [48 hours post], 14 [2 weeks post], and 17 [12 weeks post]):

Many recent studies have provided evidence implicating the immune system in a variety of neuropsychiatric disorders, including OCD. A relationship between the immune system and psychiatric disorders may be mediated through alterations of serotonin signaling. Previously conducted palliative care studies conducted have revealed a positive effect of psilocybin on the immune function. The field of psycho-neuro-immunology is predicated on reliable findings that one's psychological state of mind directly affects immune function. Studies have repeatedly demonstrated that treatment with psilocybin may lead to improvements in psychological well-being. As part of exploratory aims, if this were to happen in this patient population, it is desirable to include immune measures to correlate this finding. Alternatively, if one's psychological state deteriorates, a similar correlation would provide useful information. The specific immune measures to be included are: Cortisol, CRP, ACTH, IL-4, IL-6, IL-10, IL-12 INF-gamma, and TNF-alpha. These immune measures can be collected by venipuncture at screening, 48 hours post session, 2-week post sessions (of both active-placebo-control and the active agent, before open-label session), and at 12-week (3-month) (±3 days) follow-up.

Safety Measures (inpatient-visits 4 to 6):Columbia Suicide Severity Rating Scale (C-SSRS), Subjective Units of Distress (SUD), and vital signs including blood pressure, heart rate, and body temperature, Serious Adverse Events (SAEs), Adverse Events (AEs), spontaneously reported reactions, General Well-being, medications and changes to psychiatric medications can be collected.

Videotape/audiotape (optional): Participants are asked to allow audio-video recording of the preparatory psychoeducation and drug sessions in their entirety. These recordings are used as review aid for participants and for the therapists, and for investigators for further analysis of patient behavior, defense mechanisms, therapist interventions, manual adherence, etc. Patients can be asked to allow the study to keep these tapings indefinitely for not just for this study but also for education, future research and training purposes. Patients can be asked to initial separately audio-video taping choices in the informed consent. Patients can participate in the study in spite of refusing video recordings. The recordings can be given an identification code number, date and session number, stored in a locked file cabinet and treated confidentially.

### Research Procedures

Initial Research Procedures:

First the study, the conditions, and the procedures involved, are explained and discussed with each subject. Each subject has a chance to ask questions and to receive answers before deciding whether to participate, to be registered at CMHC, and to provide an informed consent. For safety, after completing the consent, the participants are asked to designate 2 to 3 adults who will be expected to have continuing contact with the subject, who will provide patient transportation, and who can/will observe changes in the participants' behavior and attitude over time and report back. These observers provide feedback to the research staff about the subject's behavior, mood, and attitude, in addition to the participants' self-report.

Study Research Evaluations and Procedures—Outpatient or Inpatient:

Medication and lifestyle changes may be required, including not ending nor starting new medications such as SSRIs (off recent selective serotonin inhibitors for five half-lives of the drug plus 2 weeks), abstaining from tobacco products, alcohol, or any street drugs such as marijuana. Study personnel can obtain a release to contact the participant's prescribing provider to discuss current prescriptions and medication history. This is decided on a case-by-case basis upon the discretion of the PI.

Psychotropic medications cannot be taken in the previous two weeks, with Prozac (fluoxetine) not being taken within the last 5 weeks prior to receiving any study agents. Participants may only take prn (as needed) benzodiazepines (i.e. lorazepam) up to three days before the session. Participants refrain from taking any medications the day of either the experimental or active-placebo-control treatment session, except prescribed opiate pain medication or over-the-counter non-narcotic pain medication at any time. It is also necessary to refrain from alcohol the day before, the day of, and the day after each experimental session.

Physical exam, ECG, blood and urine tests for registration and screening/CMHC/CNRU patient registration: As part of the study and the subject screening, subjects has a physical examination, ECG, and screening blood and urine tests (including comprehensive metabolic panel, chemistries, hematological tests, thyroid function tests, urinalysis, urine toxicology, and if applicable pregnancy tests). These results become part of the research case record.

Randomization and one-time Administration of the Study agent Psilocybin or active-placebo-control agent Niacin (v5) and requiring minimum of 3 overnights on CNRU (visits 3 to 6) with inpatient registration:

Psilocybin and niacin (active-placebo-control agent) are administered in identically appearing opaque, size 0 gelatin capsule with approximately 180 ml of water. The dose of psilocybin can be 0.25 mg/kg and the niacin can be 250 mg. Each participant has the option of receiving the study drug and participating in the experimental treatment sessions. In the study, half of the participants are randomized to receive psilocybin (n=15) or the active-placebo-control, niacin (n=15). Following the first treatment session of either the active agent or active-placebo-control, participants who were randomized to receive active-placebo-control can be offered the option to receive open-label psilocybin. (The blind can be broken at 48-hours to make this determination; this is after the primary endpoint. Clinical raters remain blind to the treatment assignment). Data is collected from all subjects after open-label treatment for descriptive purposes, but these data will not be analyzed as part of our primary analysis plan.

Psychiatric Evaluation, Clinical Assessments, and Study Evaluations—paper or online measures, assessments, ratings, surveys, & tasks: Online surveys and measures can be offered using secure data capture systems, e.g., Qualtrics or REDCap, or paper documents. These evaluations include standard OCD Research clinic questionnaires, clinician administered diagnostic interviews and reports, medical and clinical assessments, patient self-report measures. This testing may last up to 1 to 2.5 hours.

Research study measures, follow-ups and assessments are outlined and represent study specific clinician administered and self-administered measures. Safety measures and checks can be completed at some inpatient study visits: C-SSRS, SUD, vital signs, SAEs/AEs, spontaneously reported reactions, general well-being, medications, and changes to psychiatric medications.

All participant responses to measures and assessments can be recorded either on traditional paper formats and scanned to a Yale share drive/server, or in online data capturing systems (Qualtrics, REDCap) which instantly upload responses or surveys to a secure server. All portable digital participant data can be uploaded immediately and stored in Yale OCD Research computer databases, on password-protected computers in locked offices, and/or on secure Yale servers.

MRI/fMRI Procedures:

Structural (sMRI) and Functional (fMRI) Magnetic Resonance Imaging is performed at the Yale Magnetic Resonance Research Center (MRRC), located in the Anlyan Center, and overseen by faculty and trained MR research technologists at the MRRC. After documented, signed, informed consent, a review of the MM Safety Questionnaire, and additional screening and safety determination if necessary, participants are accompanied to the scanner by study personnel and given detailed instructions. Participants are asked to remove everything from their pockets, and to remove any potentially ferromagnetic articles of clothing, and must then pass through a metal detector according to standard safety procedures. At this time, the participants are presented to the research technologist who facilitates entry into the scanner. Total MR acquisition may last between 1.5 and 2 hours depending on acquisition of all or a subset of sequences, and whether some series must be re-acquired due to motion or other confounds/artifacts.

sMRI can be acquired in horizontal, coronal or parasagittal planes using an SPGR sequence. Images can be skull-stripped and segmented into cerebrospinal fluid, gray matter, and white matter. Volumetric assessments can be made of the major lobes of cerebral cortex and of subcortical structures, including basal ganglia.

MRI acquisition can be conducted using a Siemens MM scanner (i.e., “PrismaB,” a 3 Tesla MRI) located at the MRRC and using standard MRRC sequences already in use by other investigators. Using a 64-channel head coil (HEA;HEP), the scan protocol can begin with a 3-plane localizer, scout image, and aligned 3-plane localizer that will serve as a localization series for the other acquisitions. The remaining multi-modal magnetic resonance pulse sequences include: high resolution T1-weighted imaging (i.e., MPRAGE or FLASH); high resolution T2-weighted imaging (i.e., SPACE); spin echo field maps will be acquired in opposing phase encoding directions prior to each fMRI session; multi-band BOLD gradient echo planar imaging at rest and under challenge (i.e., task paradigm); diffusion weighted imaging (DWI), arterial spin labeling (ASL), and proton density weighted imaging (PD). The T1 MPRAGE and T2 SPACE acquisitions are set to use an “Adjust Volume” that matches the size and positioning of the spin echo field maps and resting state fMRI scans. Participants may be asked to return for a repeat session due to inadequate image acquisition or time constraints. In the case of failed MR studies, no more than three repeat sessions will be attempted. MR compatible equipment provided by the MRRC may be used to track eye movement during the MRI procedures. Illustrative sequence parameters are provided in Table 1 below.

TABLE 1
MRI Sequence Parameter Summary

| Seauence | TA | TR (ms) | TE (ms) | TI (ms) | FOV (mm) | Flip | VOX (mm) |
|---|---|---|---|---|---|---|---|
| Localizer | 00:09 | 40 | 3 | — | 300 | 15 | 1.2 × 1.2 × 5.0 |
| Scout | 00:14 | 3.15 | 1.37 | — | 260 | 8 | 1.6 × 1.6 × 1.6 |
| Localizer (aligned) | 00:21 | 104 | 3 | — | 300 | 15 | 1.2 × 1.2 × 5.0 |
| Spin Echo Field Maps | 01:04 | 8000 | 66 | — | 208 | 90 | 2.0 × 2.0 × 2.0 |
| fMRI & t-fMRI (each) | varies | 800 | 37 | — | 208 | 52 | 2.0 × 2.0 × 2.0 |
| T1 MPRAGE | 06:38 | 2400 | 2.22 | 1000 | 256 | 8 | 0.8 × 0.8 × 0.8 |
| T2 SPACE | 05:57 | 3200 | 563 | — | 256 | — | 0.8 × 0.8 × 0.8 |
| Diffusion MRI (each) | 05:45 | 3230 | 89.2 | — | 210 | 78 | 1.5 × 1.5 × 1.5 |
| T1 FLASH (40 sl) | 01:26 | 300 | 2.43 | — | 220 | 60 | 0.9 × 0.9 × 3.0 |
| Proton Density (40 sl) | 00:50 | 1000 | 20 | — | 220 | 90 | 2.5 × 2.5 × 3.0 |
| ASL (40 sl) | 06:09 | 3000 | 20 | — | 220 | 90 | 2.5 × 2.5 × 3.0 |

Pre-MRI plain radiography (x-ray) rule-out for metallic objects: a pre-MRI X-ray may be ordered in accordance with the American College of Radiology MR safe practice guidelines if participants indicate the possibility that any work in a metal shop or other areas which may have exposed them to imbedded metal.

The use of x-ray as a pre-screen for MR scans does involve exposure risk to radiation from a skull radiographic exam. The participants concerned are informed that this exposure to radiation is for research purposes only. The total radiation exposure amount of one film skull x-ray is equivalent to a uniform whole-body exposure of 12 days of exposure to natural background radiation. This use involves minimal risk and is necessary to obtain the information required to participation in a MR scan.

For a skull radiographic (1 film) exam, the expected dose to a patient would be the following Total Effective dose: 10 mrem or 0.1 MSv.

Dose to breasts: 1 mrad Dose to red marrow: 20 mrad Dose to thyroid: 40 mrad Dose to uterus: 1 mrad Dose to ovaries: 1 mrad

Neuropsychological—Cognitive Assessment. Data is gathered utilizing various computer/tablet tasks and some general assessments to explore subjects' cognitive, cognitive performance and executive functioning. The general intellectual functioning of participants can be assessed to provide a basis of comparison. Subjects are seated in front of a computer screen or given a tablet. Subjects are presented with a series of visual and auditory stimuli. The testing may last up to 2 hours.

Blood Draws for Research:

Immunological Panels/Measures—Participants are asked to immunological panels drawn (3 tubes, less than 50 mL). The specific immune measures to be included are: Cortisol, CRP, ACTH, IL-4, IL-6, IL-10, IL-12 INF-gamma, and TNF-alpha. For the randomization phase (RP), these are collected by venipuncture at screening (v2), 48 hours post session. For the open label (OL) phase, the immune measures are collected at 48 hours post open label.

Genetic Testing/Repository (option): Participants can be asked to provide specimens for future genetics blood testing. The purpose of this bloodwork repository is to create a resource for current and future studies of the genetics of OCD, of genetic predictors of treatment response and the relevance of cytokines in the pathophysiology of OCD. Future research may examine genes (the units of inheritance that are passed down from generation to generation), including finding out the details of how a participant's DNA is put together, such as whole exome or genome sequencing, or genome wide association studies (that is, looking at genes other than those associated with a specific disease).

Follow-up Appointments: Ratings and evaluations can be conducted from 1 day post (v 6) to 12 weeks/3 months (v12) post administration of the study medication to gather ratings and data about the participant's experience and to document any clinical changes. The ratings portion of the follow-up appointments can be aimed at obtaining the needed study data.

Post Unblinding and 48 hour Post ratings: The integrative session of the follow-up appointments can be aimed at providing opportunities for discussion of the participant's experience during the drug administration. With a designated investigator, the participant can be encouraged to discuss his/her thoughts and feelings that arise and to help integrate this information into his/her life.

Out of State Participants: Some participants live in another state and it may not feasible to have them come to the CMHC for an in-person follow-up visit. In this case the follow-up visits can be conducted via telephone or a secure video communication platform (i.e. Zoom, Skype, etc.).

Due to the COVID-19 pandemic it is wished to expand this option to local participants as well. This allows for minimizing in person contact which will reduce the risk of potential exposure to COVID-19.

Audio-Video Recording—initial study-experience discussions and drug administration (v4, v5, v6) can be audio and video (or digitally) taped for review by the investigator-therapist and by the participant. Tapes (or digital recordings) can be coded with study number and date (accessible only to the research staff) but otherwise not labeled. If the participant is willing and allows, then the coded tapes can be kept indefinitely for ongoing educational, future research and training purposes.

Full Study Procedure Schedules are Outlined in the Randomized Phase

Baseline Study Procedures and evaluations: Participants present to CMHC/Yale OCD Clinic for the evaluation, the study procedures are described, and the subject's questions are answered prior to obtaining an informed consent. All study inclusion and exclusion criteria are checked during by the principal investigator and the entire research team, and still eligible adult patients are scheduled for the study baseline physical and other evaluations. The investigator or research staff member obtaining consent ask the participant to provide a brief summary of the study to ensure they understand what is being asked of them and any potential risks and benefits. Once consent is obtained, subjects participate in the following procedures: a biopsychosocial interview, psychological evaluation, a brief cognitive evaluation, a physical examination, and a standard liver panel taken prior to the study psilocybin session that ensures that the patient possesses sufficient liver function to metabolize psilocybin.

If a female subject is pregnant, breast-feeding, intending to become pregnant, or does not agree to use an approved birth control method (i.e., oral, injectable, or implant birth control, condom, diaphragm with spermicide, intrauterine device, tubal ligation, abstinence, or partner with vasectomy) she is excluded.

The participant fills out an MRI safety data sheet. If the participant is determined to be eligible, they are scheduled for their study visit. The duration of this visit does not exceed 4 hours.

The participant has at least two introductory sessions with the PI, Co-I, and/or key study personnel in which the study, the scheduling and the procedures are reviewed. All participants' questions are answered prior to obtaining an informed consent. The 4 days/3 nights inpatient study dates (v3 to 6) are thoroughly discussed with the participant. These discussions will include the initial neuropsychiatric testing and any additional study psychoeducation sessions needed will be scheduled with the PI or one of the Co-Is.

This clinical and medical information are reviewed by the research staff for any other contraindications to research participation. Subjects who are found to be abusing controlled substances are excluded from the study, and do not receive any economic compensation for their participation in the screening process. Subjects who, in the opinion of the covering study medical doctor, are eligible to continue with the protocol procedures (after the results of baseline measures and diagnostics are considered) initially are admitted to the CMHC/CNRU as an outpatient, while preliminary study assessments are completed at the second visit. ECG, breathalyzer and blood samples for routine testing. A urine sample can be collected for urine toxicology, pregnancy test, and urinalysis.

Eligible participants are scheduled and then later admitted to the CNRU at CMHC as an inpatient for 3 nights/4 days (Randomization phase visits 3, 4, 5, 6), surrounding the initial drug administration (or more, at the option of the subject and the investigator).

Study Visits 3 to 7/CNRU Inpatient Admission & Discharge to Outpatient Follow-Up:

Study Visit 3/CNRU inpatient admission—overnight/ongoing evaluations—During the third visit, the participant is admitted as an inpatient on the CNRU and vitals will be taken. This allows the participant to adjust to the CNRU and to get used to sleeping over. And it allows the unit to conduct registration paperwork, and any labs required (if not already completed as an outpatient).

The participant is encouraged to bring some personal possessions (photos, pictures, writings) with them to enhance their experience with the unit and the treatment session (v5). The participant is administered the study-specific measures and assessments by a trained clinician. A battery of study self-reports are administered during visits 3 to 4. Participants also complete neuropsychiatric evaluation/testing and any additional education (v3-4) about the experimental session prior to the drug administration (v5).

Visit 4/inpatient-overnight/fMRI/taping of discussion—after the initial neuropsychiatric evaluations are complete and the subject has been admitted as an inpatient to the CMHC/CNRU. On the day before drug-administration the participant have a pre-drug session fMRI and complete designated assessments. If not already completed, the participant completes vitals and provide a urine sample or drug and pregnancy testing. Vitals are continued to be taken during inpatient study days (v4, v5): Blood pressure, heart rate, and temperature; suicidal ideation, subjective distress, and medications are reviewed, with any changes noted.

The participants present to the Anlyan MRRC for their first pre-drug MRI Scan. While in the scanner, participants will rest quietly for 10 minutes for the collection of resting-state data; the total time inside the scanner will be ˜120 minutes. Once the MRI scan is completed, participants are accompanied by research staff back to CMHC where they will again stay overnight.

Participants meet with investigator-therapists to discuss life, goals, desires, beliefs, and past experiences, to be introduced to mindfulness techniques, spiritual cognitive-behavioral stress management and to address specific hopes, fears, and goals for the upcoming treatment sessions. Audio-video recordings (with coded id number) of this session are made to allow the participant and the investigator-therapists to review the session discussion.

Visit 5/Inpatient, Randomization to Study Medication or Active-Placebo Control/Experimental Treatment Session

Randomization: On either the day before (v4) or the day of drug administration (v5), the participants are randomly assigned to one of the two conditions, experimental intervention (psilocybin 0.25 mg/kg) or active-placebo-control (niacin 250 mg). The rationale for using niacin as the active-placebo-control agent rather than active low dose of psilocybin (ranging 2 mg-4 mg), which have been used in the recent palliative care studies and OCD study, is because a partial response has been reported even with these low doses. Psilocybin acutely attenuated OCD symptoms even after low doses.

The randomization is handled by the designated staff, who are the only personnel with access to the psilocybin. Both the active and placebo are prepared by designated study staff by a compounding process. The condition assignment is maintained from study enrollment up through the course of the study, as there is no crossover design. If there is an adverse event or other emergency requiring knowledge of participant's condition assignment, as when pharmacological intervention is necessary, the blind may be broken for an individual participant.

Study preparation the day of drug administration (v5): the CMHC/CNRU staff wakes the participant, begins study preparation and serves breakfast. The participant is allowed to eat only a light breakfast of hot cereal or bread/toast on the day of the session. Caffeinated clear liquids or juices are permitted, but no milk as drug absorption might be affected.

At approximately 10:00 am, the experimental session begins. The participant is settled into a study treatment room at CMHC on CNRU. The research staff monitors the participant's vital signs at 15 minutes and thirty minutes post drug administration, then every thirty minutes thereafter for the first two hours. Vitals monitoring continues at least hourly during the rest of the experimental session and then at regularly scheduled CNRU shift checks for the balance of the inpatient stay. The prepared study medication is delivered by key research staff. The experimental medicine is administered between 10:00 and 11:00 hours. Psilocybin or niacin is administered in identically appearing opaque, size 0 gelatin capsule with approximately 180 mL of water. The dose of psilocybin is 0.25 mg/kg and the niacin is 250 mg. (Each participant has the option of participating in the experimental treatment session). In the study, half of the participants are randomized to receive psilocybin and half the niacin.

The session duration is expected to last up to 6 hours under supervision from the PI, and/or key research personnel. The key study personnel who administer the questionnaires remain blind as the participant's condition assignment. Participants are expected to remain under observation until both the participant and investigator agree that their perception, cognition, functioning and judgment are no longer impaired by the drug.

Visit 5 Drug Administration—Schedule Administered by Trained Research Staff and/or a Physician

Participants are encouraged to lie comfortably in bed wearing eye shades during the first few hours of the experience and to listen to pre-selected music chosen by the study staff. Two team members remain with the participants the entire time and are available for any processing of intrapsychic material made manifest by the treatment. In addition, the continued need for conventional medications (e.g. non-opioid analgesics such as ibuprofen) to alleviate pain is monitored in a systematized, ongoing manner. Participants are asked about any residual side effects of other adverse events (AEs) and any appropriate referrals for treatment is made as necessary. They also have their vitals taken. Participants are debriefed by the PI and/or research staff.

Participants are asked to remain at CMHC overnight on the evening of drug administration for monitoring and study assessments. They have the option of being discharged the following day after examined and medically cleared by the PI or another medical doctor. However, if a participant has traveled from out of state (or from a distance that is inconvenient to return the following day for an fMRI scan), he/she is offered an option to stay overnight at CMHC. Driving will be performed by a family member or friend, but not the participant. Alternatively, a taxi can be provided to the participant.

If participants experience or report on any adverse effects or if the research staff feels that at the end of testing, that the participant has any adverse effects, participants will be asked/required to remain until effects wear off and medically cleared by the PI or another medical doctor. Medical assistance can be offered. The investigators can query participants about suicidal ideation or intent during screening, and prior to each psilocybin session. Participants who evidence suicidal ideation or intent can be withdrawn from the study and referred to appropriate psychiatric care.

In the event that participants experience distressing anxious symptoms during the drug session, the clinical personnel in attendance (the principal investigator and/or research team members) can initiate verbal communication designed to reorient and reassure that participant. The occurrence and intensity of anxiety or panic responses can be reduced through providing participants with information on potential drug effects, supervision and monitoring of participants for the duration of drug effects and using ascending dose designs. The goal in this study is to treat participants undergoing anxiety or panic reactions first by verbal and psychological interventions and using anxiolytic medication only after verbal and psychological interventions have failed, and if participants are endangering themselves or others. In case of insomnia or an overly anxious reaction, the investigator may prescribe a benzodiazepine or zolpidem as a “rescue medication” for the day or night after drug administration. Residual symptoms can be addressed during the frequent follow-up psychotherapy visits with the investigators.

Rescue Medications, Treatments, and Procedures: The PI may prescribe a designated rescue medication in the event of symptoms that require it during or after the experimental session. Rescue medications may be a benzodiazepine, zolpidem, or other anxiolytic or sedative according to the physician's clinical judgment.

Should a longer psychiatric hospitalization be necessary, then participants can remain admitted to the study unit. The hospital can be informed in advance about the study to arrange for patient registration. Participants can be free to withdraw from the study whenever they wish, but they have to be medically cleared before being discharged to home. In addition, if any participant presents with an adverse reaction to the psychopharmacological agent as reported by them or assessed by a member of the research team, the study can be discontinued, and medical assistance can be called for. Finally, the participant may be withdrawn from the study if his/her withdrawal is in the best interests of either the participant or of the research (e.g., due to a medical condition making the procedures unsafe or any condition making interpretation of the results difficult).

Visit 6, Inpatient 24 hours post-drug administration/possible discharge—Participants complete some additional assessments, including an A-YBOCS examining specific OCD symptoms over the past 24 hours, will be followed-up with the PI, Co-I, and/or key personnel (v6), and reminded to present for the second fmri appointment the next day. Then the participant may be discharged from the inpatient stay into the care of a community/family supporter (to provide observation and transportation) once medically cleared. After participants return home, the researchers check at study visits or by telephone each day to inquire about how the participant is feeling, until the one-week post administration visit (V8).

Visit 7 Outpatient 48 hours post dose—Evaluation, fMRI, Unblinding: Participants must have someone drive them to and from this visit. Participants present to the CMHC for the second MRI scan and complete clinical assessments, A-YBOCS, and a brief cognitive assessment. The immune measures would again be collected by venipuncture at this 48-hours post session.

The participant is debriefed again and given the chance to discuss his/her experience with the study. The participant continues to receive follow-up support and ratings 1 week, 2 weeks, 4 weeks, 8 weeks, and 12 weeks after drug administration, with the PI, Co-I, and/or key study personnel. During non-drug and experimental sessions, participants are asked to think about and discuss their thoughts and emotions about carrying a diagnosis of OCD, including anxiety and the impact the OCD has had on their lives. They may experience emotional responses to speaking about these thoughts, feelings and concerns.

The blind can be broken (48 hours post administration—the primary endpoint) at this visit. Afterwards, the participants who were randomized to receive the active-placebo-control agent, can be offered the option to receive open-label psilocybin. After participants return home, the researchers check at study visits or telephone subjects every day for a week to inquire about how the participants are feeling. They are re-screened not later than a week after the blind is broken to make sure they still meet study criteria. The open-label phase of the study can include optional MM scans.

Outpatient follow-up support and ratings for the randomized phase continue for about 3 months at designated times (v8, v9, v10, v11, v12). Community observer ratings are obtained at week one and week 12. Assessments will continue as outlined, post-drug weeks 1 to 12 (visits 8 to 9). The immune measures would again be collected by venipuncture at 48 hours post session (of both active-placebo and the active drug agent, before open-label session). The specific immune measures to be included are: Cortisol, CRP, ACTH, IL-4, IL-6, IL-10, IL-12 INF-gamma, and TNF-alpha.

Study Schedule for the Open Label Phase:

Open Label Psilocybin Phase—The participants who choose to receive open-label psilocybin are re-screened after the blind is broken during the experimental phase (d 10) to make sure they still meet study criteria. If they are eligible and still choose to enroll in the open-label continuation, they may do so, undergoing a psilocybin-assisted session.

The open-label phase of the study follows nearly identical procedures to those used in the randomized phase. Participants are re-assessed on all study measures following a similar schedule to the randomized phase. The immune measures would again be collected by venipuncture for the open-label session (v 12, 48 hours post-drug).

Like the first phase, participants in the open label phase are offered MRI Safety Questionnaires and fMRI scans on the day before drug administration and 48 hours post drug administration. Participants need not complete any portion of the open-label phase or the several follow up support appointments for their participation in the study to be considered completed.

Data is collected from all subjects after open-label treatment for descriptive purposes and is viewed not as part of our primary analysis plan but as part of a secondary analysis examining the pharmacological effects on OCD symptomatology.

### Risks of Study:

Potential risks of psilocybin: Safety Pharmacology Studies in humans and nonhuman animals indicate that psilocybin has very low toxicity. The LD₅₀ ranged from 285 mg/kg in rats and mice, cited in Merck Manual, 19th edition). The maximum tolerated dose of psilocybin in humans has not been defined or established. The best estimate of a lethal dose for pure psilocybin in humans is about 19 grams. The full dose of psilocybin that will be administered in this study is over 100 times smaller than LD₅₀ in nonhuman animals and in humans. A Phase I trial of psilocybin at oral doses of up to 0.6 mg/kg demonstrated no serious adverse events. Psilocybin is not associated with disease or damage to any organ or system. More commonly, damage or disease to organs (as renal failure) is associated with mistakenly consuming poisonous mushrooms under the belief that they are psilocybin-containing mushrooms. Three cases of death possibly related to the direct toxic effects of Psilocybe mushrooms (the natural source of psilocybin) have been reported in the world literature. One fatality occurred in a post-cardiac transplant patient who ingested Psilocybe mushrooms. It is not known however whether psilocybin was the proximal cause of death or whether death occurred secondary to the cardiac stimulant phenethylamine, which is also present in the mushrooms. In the French literature, another fatality linked to Psilocybe mushroom use was associated with far higher blood concentrations of the active metabolite of psilocybin than would be expected with the doses proposed in this study.

A final fatality from Psilocybe mushroom ingestion was poorly characterized, and no psilocin concentrations were reported. In a review of other adverse effects of Psilocybe mushrooms, an additional 10 individuals were reported to have experienced accidental deaths associated with Psilocybe mushroom ingestion. In most cases, these accidental deaths resulted from falls from buildings. Given the long and uncontrolled use of Psilocybe mushrooms in various societies and cultures, that there are only 3 known fatalities at best indirectly linked to the physiologic effects of mushroom ingestion is an argument for the general safety of this natural product. It is believed that the use of pure psilocybin will decrease the risk of cardiac toxicity by avoiding any additive or synergistic effect [symbol] of phenylethylamine. Further, a recent review of the potential harm of the ingestion of Psilocybe mushrooms by the Dutch medicinal advisory board found that the risk of acute or chronic toxicity was low.

To date, over 2000 participants have received psilocybin under controlled conditions in scientific studies with no reports of the occurrence of a significance of adverse events deemed to be associated with drug administration. The most commonly reported adverse events from the scientific literature are psychological in nature and include the induction of negative emotional states and paranoid/delusional thinking during psilocybin sessions, as well as far less frequent reports of Hallucinogen Persisting Perception Disorder (HPPD). A review of studies conducted worldwide between 1999 and 2008 identified only one subject (out of 110) who experienced any persistent perceptual symptoms associated with HPPD and these symptoms were mild, brief, and resolved within three days of psilocybin exposure. Rates of prolonged psychiatric symptoms of any kind following psilocybin exposure in healthy study participants are estimated to be 0.08-0.09%. As in the phase 2 studies, common physical adverse events associated with psilocybin administration include increased BP and heart rate, nausea, and headaches. In the phase 2 studies of cancer related anxiety and depression no cases of HPPD were identified and no participants developed any symptoms of paranoia or anxiety that required pharmacological intervention or anything more than reassurance from session facilitators. In a survey of 1993 individuals in the context of illicit use of psilocybin-containing mushrooms, 11% put self or others at risk of physical harm. Of the respondents, 2.6% behaved in a physically aggressive or violent manner and 2.7% received medical help. Of those whose experience occurred >1 year before, 7.6% sought treatment for enduring psychological symptoms. 3 cases appeared associated with onset of enduring psychotic symptoms and 3 cases with attempted suicide.

To date, there have no reports of physical harm in patients who have received psilocybin under controlled conditions, and no fatalities have been associated with its use in a controlled clinical trials. The difference in the side-effect profile between the unknown doses of psilocybin-containing mushrooms consumed in an uncontrolled setting and dose-controlled medically prepared oral psilocybin consumed in a controlled setting is thought to be in part doe the focus on the set and setting of the experience. The set refers to the emotional/cognitive/behavioral state/mindset and expectations of study participants just prior to psilocybin exposure and setting refers to the physical environment in which the exposure occurs.

There are no confirmed reports of an overdose of pure psilocybin. In the United States, use of chemically synthesized psilocybin does not occur. Although psilocybin in the form of mushrooms is sometimes used non-medically, medical emergencies due to psilocybin mushrooms are very rare (psilocybin is mentioned in only 0.1% of drug-related emergency department visits). It can be anticipated that an overdose of psilocybin might present in a manner similar to “serotonin syndrome”. Removal of any residual gastric drug, supportive care, and cautious administration of a serotonin antagonist such as risperidone are reasonable interventions; however, to date no cases have described similar responses with respect to psilocybin, and the doses of psilocybin that might provoke these physiological effects would be many times greater than even the highest doses used in early human studies.

Common Side Effects

Common acute side effects of psilocybin are almost all psychological, and include anxiety, changes in thought (experiencing thinking speeding up or slowing down), changes in motion perception, changes in time perception (time slowing down or speeding up), depersonalization (feeling as if one is “outside oneself”), derealization (feeling as if the world is unreal or as if one is “in a dream”), dizziness, fatigue, impaired concentration, inattention, mood lability (rapid and sometimes profound changes in mood), nausea, nervousness, parasthesias (strange bodily sensations or feelings), perceptual alterations (general), altered time perception, alteration in visual perception (distortions, illusions and imagery seen with eyes open or closed), and unusual thought or feelings about the self or the world. Most of these effects are acute and last no longer than six hours. For the most part, people receiving psilocybin maintained insight concerning the nature and source of their experience. However, some participants occasionally exhibited paranoid ideation or temporarily lost insight into the experimental situation.

Psilocybin produces slight sympathetic system activation. Physiological effects include pupillary dilation and detectable but moderate increases in blood pressure or heart rate. Researchers sometimes detected changes in blood pressure and heart rate only at one point in time, as sixty minutes after drug administration. These effects are transient and are generally gone approximately six hours after drug administration. People in previous human studies have tolerated doses of psilocybin equal to or greater than the doses employed in this study. Researchers studying the effects of 30 mg psilocybin took precautions similar to the ones we will use in this study and no reactions requiring pharmacological intervention occurred.

Acute Side Effects

The most likely potential adverse effect of psilocybin is anxiety, or possibly panic, delusion, and cognitive impairments, particularly at higher doses (>25 mg po) during the period of acute drug action. In recent studies of psilocybin in healthy humans conducted over the past 15 years no adverse reactions other than the expected occasional episodes of fear or anxiety during the time of drug effect have been reported. Such transient episodes of fear or anxiety respond well to reassurance and have not required pharmacological intervention. Some individuals have been reported to develop paranoid and psychotic states while under the influence of psilocybin.

Insomnia: Although rare, insomnia following psilocybin administration has been reported in the literature; it has been described as self-limiting and responded well to reassurance from the study team. In the event that a participant experiences insomnia, a study physician may initially prescribe a sleep aid (i.e. diphenhydramine or hydroxyzine) and if the initial reassurance, education about sleep hygiene and pharmacological intervention was unsuccessful, a one-time dose of short-acting benzodiazepine (specifically lorazepam) and/or zolpidem can be considered.

Prolonged Side Effects

In addition to fear, panic, and paranoia during the drug session, there is the possibility of prolonged adverse psychological reactions, such as psychosis and depression. It is generally believed that individuals with previous psychiatric conditions, particularly psychosis or mania, are at an increased risk for hallucinogen-induced psychosis. Furthermore, the risk that, in vulnerable individuals, psilocybin could precipitate enduring psychiatric illness such as schizophrenia is very low, but cannot be excluded completely. For this reason, all potential participants with personal or family histories of Schizophrenia, Psychotic Disorder, or Bipolar I or II disorder are to be excluded from participation. Nevertheless, although the risk is believed to be minimal, it is possible that some participants may develop a brief psychotic reaction while under the influence of psilocybin.

The low rate of enduring psychological symptoms is consistent with a summary of such effects from 110 psilocybin research participants reported in a meta-analysis. In that report, 7 participants endorsed negative changes in psychological well-being, but only 1 participant (0.9%) reported a level of distress sufficient for him to contact the researchers. Those symptoms resolved after a few sessions with an experienced psychotherapist.

Johns Hopkins recently completed an internet survey of hallucinogen users who reported having a challenging experience after taking psilocybin mushrooms. The rates and severity of both acute and enduring problems shown in the survey are notably higher than those observed in laboratory research studies involving administration of high doses of psilocybin to carefully screened, well-prepared, and closely monitored volunteers. This observation appears to support the efficacy of the standard screening, preparation, monitoring, and follow-up procedures designed to minimize the possibility of any enduring problems.

Benefits: This study evaluates the use of 5-HT2A agonist for OCD. A greater understanding of the neurobiology involved in OCD and the mechanism of anti-obsessive action could provide an impetus for development of more effective and rapid treatments for this debilitating disorder.

Potential risks of Niacin (active-placebo-control agent): A participant's OCD might change or perhaps worsen during the course of the study (as symptoms might change during a normal course). There is no reason to believe that niacin as an active-placebo-control would exacerbate OCD symptoms. Niacin has some expected physiological side effects which is why it is used as an “active placebo”. These include flushing, tingling, itching, and redness of the face, arms, and chest, as well as headaches. Other minor but expected side effects include upset stomach, intestinal gas, and dizziness. Participants will be informed about these potential side effects and they will be monitored throughout the drug administration visits.

Risk in Changes or delay in treatment: Participants are asked to refrain from starting new medications during the study procedures. Should circumstances warrant a change in treatment, then participants are asked to immediately notify the study team.

Risk of involuntary hospitalization for patient safety: As part of enrollment in this study, participant must agree to staying overnight at the CNRU for up to 4 nights for evaluations and for monitoring after receiving psilocybin. If participant is not compliant with the requirement or it is deemed a safety concern, then there is possibility that the participant may be involuntarily hospitalized against his/her will. Also, if there is concern about participants being a risk to themselves or others during the study, there is potential for participants to be hospitalized against their will. Participants are monitored and discharged only after study physician believes that it is safe to do so.

Risks of Emotional Responses, Discomforts During Administration & follow-up meetings: During non-drug and experimental sessions, participants are asked to think about and to discuss their thoughts and emotions about carrying a diagnosis of OCD, including anxiety and the impact the OCD has had on their lives. They may experience emotional responses to speaking about these thoughts, feelings and concerns. The drug session takes place at CMHC and is staffed by two investigators. The PI and/or investigators who are present during the drug session meet the patient several times prior to drug administration. This is done as part of psychoeducation, discussing several procedures in coping with anxiety and distress should it be necessary during the drug session.

Study Endpoints

Primary Endpoint

Primary endpoint assesses OCD symptoms measured using YBOCS at 48 hours post drug administration. YBOCS are administered at evaluation, 1 day prior to drug administration, 1-day post-drug, 2 days' post-drug, 1-week post-drug, 2 weeks post-drug, 4 weeks post-drug, 8 weeks post drug and 12 weeks post-drug.

Secondary Endpoints

Secondary outcome measures look at the effect of psilocybin on symptoms depression, quality of life, beliefs about spirituality and self-boundaries, community observer-rated changes in participant behavior and attitudes, mystical experiences, personality dimensions, symptoms of perception.

In addition, a secondary objective of this study is to determine the feasibility of administering psilocybin to this patient population, with regards to the following issues: safety, patient recruitment, and retention. The duration of the proposed study will be long enough to administer the drug one time to each of the thirty participants and to conduct follow-up assessments.

Exploratory Endpoints

With respect to associations between resting state, functional and structural MRI in OCD and symptom clusters or treatment outcome variables, the study will initially focus on connectivity abnormalities that have previously been described by applicants and others: altered global brain connectivity in the basal ganglia, increased seed-based functional connectivity between ventral striatum and medial prefrontal cortex, and increased coherence between default and attentional networks.

A fully data-driven analysis is performed to identify regions beyond these initial hypotheses where changes in global brain connectivity correlate with clinical change after psilocybin treatment.

The short-term and continued effects of psilocybin-assisted therapy on immune function are investigated. Changes in immunological function are assessed at baseline, 48 hours after drug session, 2 weeks after drug session, and 12-weeks after drug administration.

### Example 2

A double-blind, placebo controlled study was conducted of psilocybin in OCD (NCT03356483). 9 out of a planned 30 patients with treatment resistant OCD have been randomized to either a single dose of psilocybin (0.25 mg/kg) or active placebo (niacin 250 mg). Psilocybin is administered in a controlled setting in an inpatient research unit in the presence of two research staff members who provide support during drug administration. Structured psychotherapy is not provided. The primary clinical endpoint is at 48 hours, though follow-up data is collected for 3 months. OCD symptoms are measured with the acute Yale-Brown Obsessive-Compulsive Scale (acute-YBOCS). Patients complete two resting-state fMRI scans, one 24 hours before treatment and the second 48 hours after treatment. In addition, numerous questionnaires are collected designed to examine how the phenomenology of subjective experiences during psilocybin intoxication relates to clinical changes.

Results: OCD patients who received psilocybin (N=5) exhibited reductions in acute-YBOCS scores from baseline (M=25.5, ranging from 18 to 29) to 48-hours (M=15, SD=11.52,) after drug administration (1-tailed within-subject t-test: t(3)=2.71; p=0.036). Among the OCD patients who received placebo (niacin) (N=4), acute-YBOCS scores minimally changed from baseline (M=25.5) to 48-hours (M=23.5) after drug administration. Paired t-tests could not be completed for this group due to small sample size. Three patients (N=3) who were randomized to placebo returned two weeks later to receive open-label placebo; both had robust and enduring clinical responses.

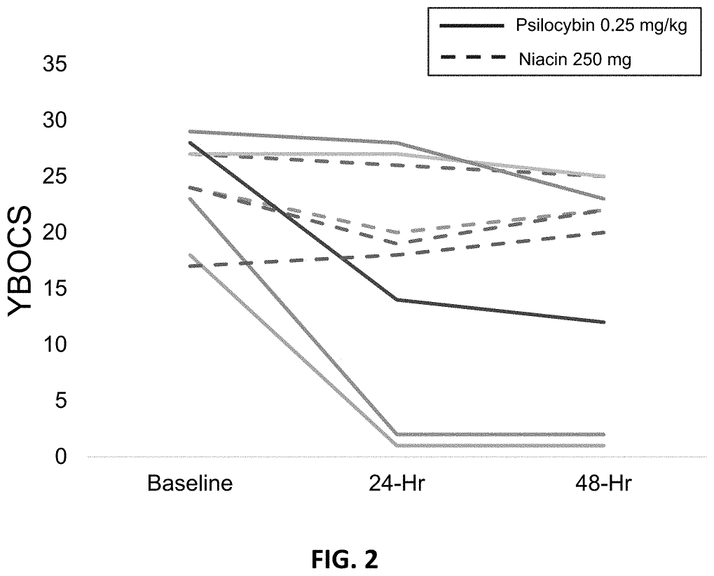

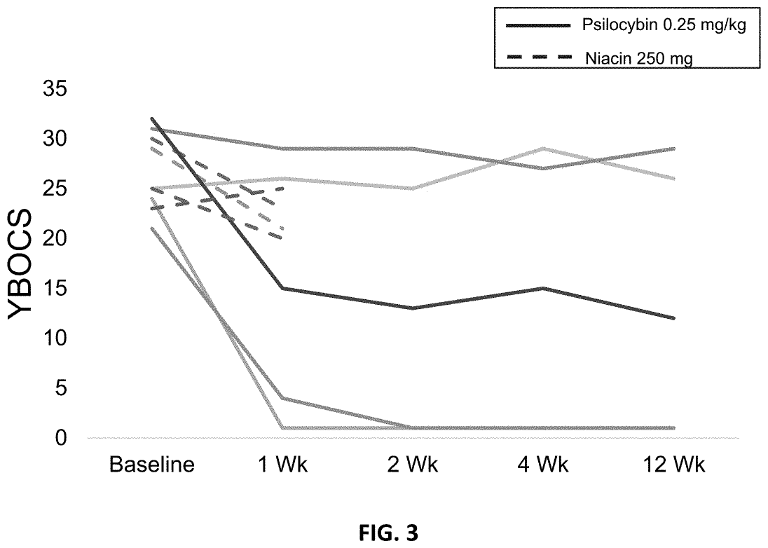

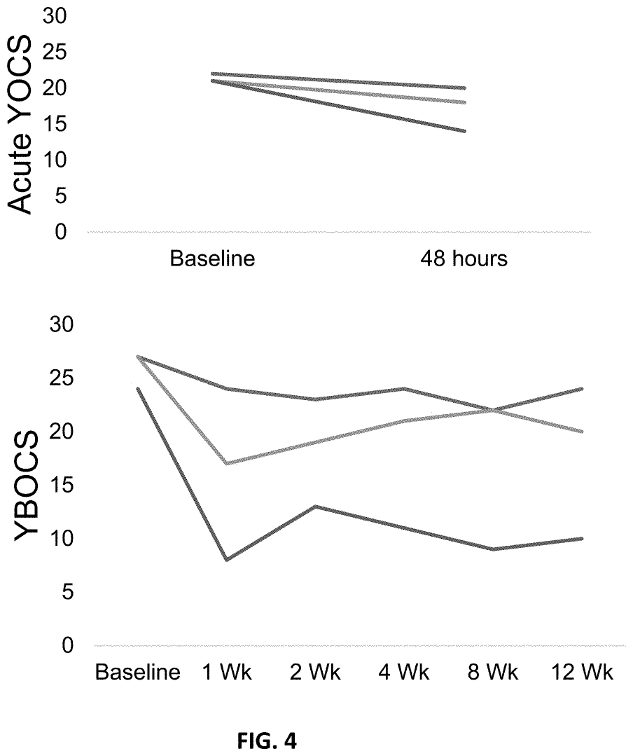

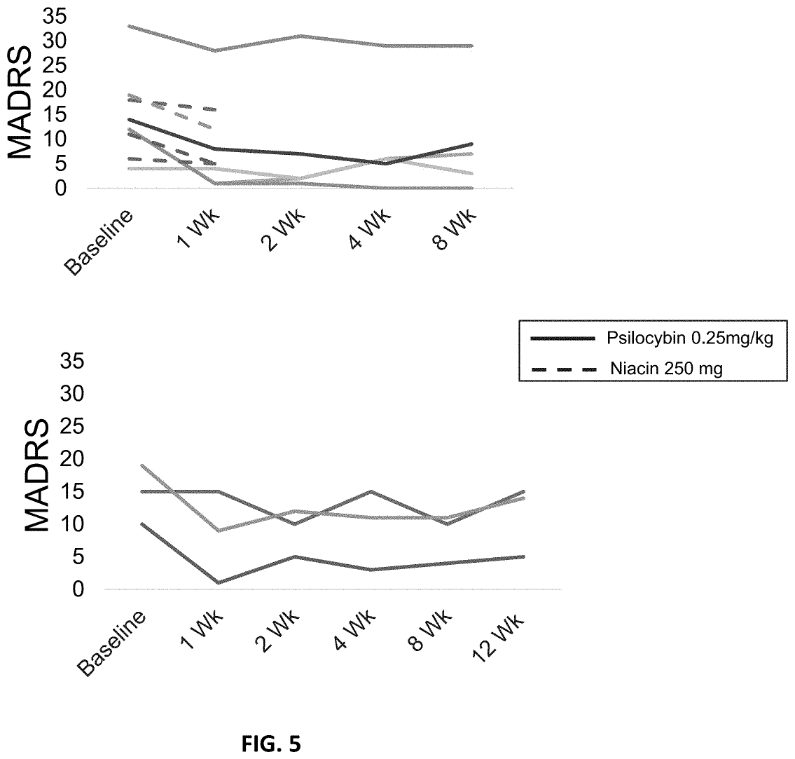

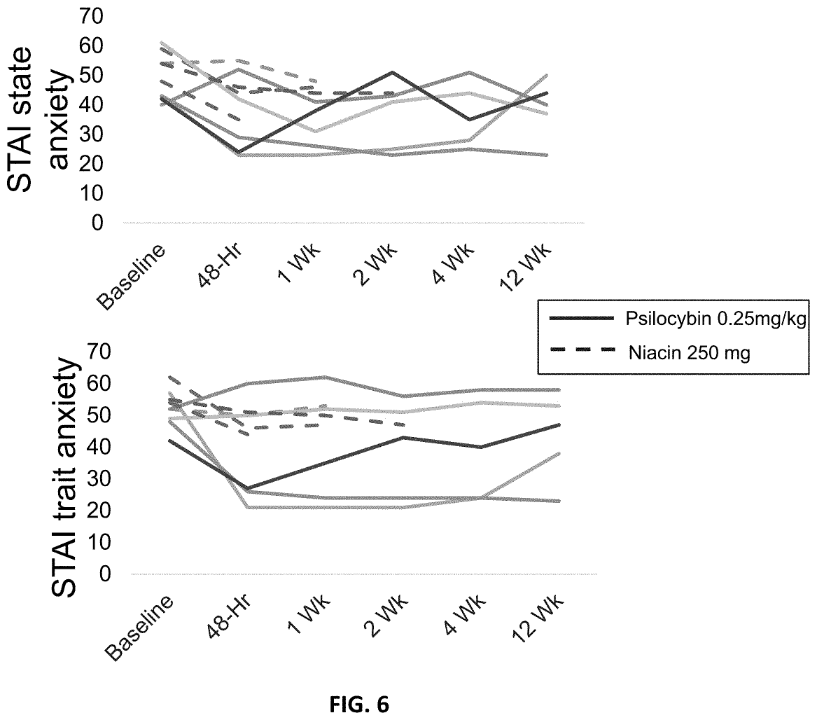

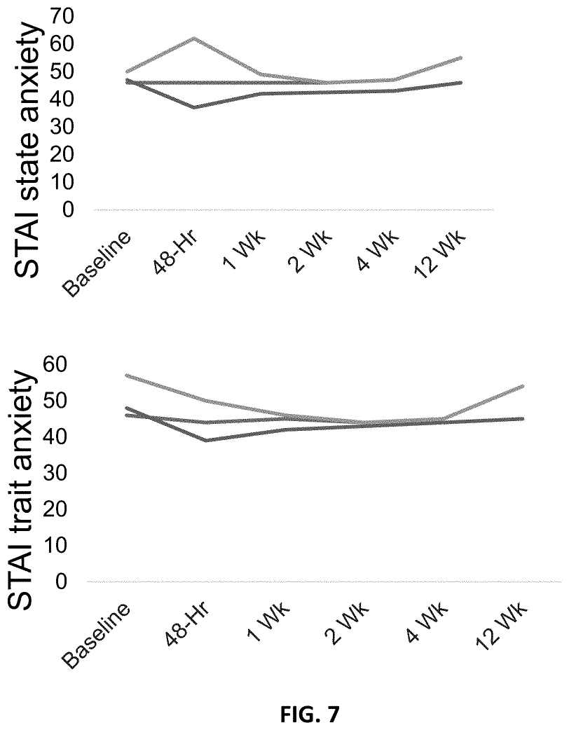

FIG. 2 is a graph of acute YBOCS over time for psilocybin and placebo. FIG. 3 is a graph of YBOCS over time for psilocybin and placebo. Of those subjects receiving psilocybin, three subjects exhibited a robust improvement in symptoms. The improvement in symptoms seen in these three subjects unexpectedly persisted for 12 weeks. One of these three subjects showed a robust and lasting improvement in symptoms, FIG. 4. As shown in FIG. 5, although depression scores were not high in most subjects; they improved after psilocybin in several subjects (MADSRS is the Montgomery-Asberg Depression Rating Scale). As shown in FIG. 6, state anxiety scores improved in those subjects whose OCD symptoms also improved following treatment with psilocybin. In contrast, as shown in FIG. 7, no changes in anxiety were noted in these placebo-dosed subjects.

### Enumerated Embodiments

The following enumerated embodiments are provided, the numbering of which is not to be construed as designating levels of importance:

Embodiment 1 provides a method of treating, preventing, and/or ameliorating obsessive-compulsive disorder (OCD) in a subject, the method comprising:

administering a therapeutically effective amount of a composition comprising a 5-HT receptor agonist to the subject, wherein the subject:
i) is suffering from OCD;
ii) is not diagnosed with obsessive-compulsive personality disorder (OCPD); and

reducing, reversing, and/or eliminating OCD symptoms in the subject.

Embodiment 2 provides the method of embodiment 1, wherein the 5-HT receptor agonist is psilocybin, or a salt, solvate, analog, or functional equivalent thereof.
Embodiment 3 provides the method of any one of embodiments 1-2, wherein the effective amount of psilocybin, or a salt or solvate thereof, is a dose of about 0.05 to about 0.55 mg/kg.
Embodiment 4 provides the method of any one of embodiments 1-3, wherein the effective amount of psilocybin, or a salt or solvate thereof, is 0.25 mg/kg.
Embodiment 5 provides the method of any one of embodiments 1-4, wherein the therapeutically effective amount is administered in a single dose to the subject.
Embodiment 6 provides the method of any one of embodiments 1-5, wherein the reduction, reversal, and/or elimination of OCD symptoms in the subject persists for at least 8 weeks after administration.
Embodiment 7 provides the method of any one of embodiments 1-6, wherein the subject was not treated with a contraindicated medication for at least 4 weeks before starting treatment with the 5-HT receptor agonist.
Embodiment 8 provides the method of any one of embodiments 1-7, wherein the reduction, reversal, and/or elimination of OCD symptoms in the subject is measured by the Yale-Brown Obsessive Compulsive Scale (YBOCS) score.
Embodiment 9 provides the method of any one of embodiments 1-8, wherein the subject has a pre-treatment YBOCS score of at least about 19.
Embodiment 10 provides the method of any one of embodiments 1-9, wherein one week after the administration of the 5-HT receptor agonist the subject has a YBOCS score that is at least about 25% smaller than the pre-administration YBOCS score.
Embodiment 11 provides the method of any one of embodiments 1-10, wherein the reduced YBOCS persists for at least about 8 weeks after administration.
Embodiment 12 provides the method of any one of embodiments 1-11, wherein the method further includes the step of reducing, reversing, or eliminating anxiety.
Embodiment 13 provides the method of any one of embodiments 1-12, further comprising the step of administering a maintenance dose of the 5-HT receptor agonist upon symptom recurrence.
Embodiment 14 provides a method of treating, preventing, and/or ameliorating obsessive-compulsive disorder (OCD) in a subject, the method comprising:

administering a therapeutically effective amount of a composition comprising a 5-HT receptor agonist to the subject, wherein the subject:
i) is suffering from OCD;
ii) is not diagnosed with obsessive-compulsive personality disorder (OCPD); and

reducing, reversing, and/or eliminating obsessive thoughts and/or compulsive behaviors in the subject.

Embodiment 15 provides the method of embodiment 14, wherein the 5-HT receptor agonist is psilocybin, or a salt, solvate, analog, or functional equivalent thereof.
Embodiment 16 provides the method of any one of embodiments 14-15, wherein the effective amount of psilocybin, or a salt or solvate thereof, is a dose of about 0.05 to about 0.55 mg/kg.
Embodiment 17 provides the method of any one of embodiments 14-16, wherein the effective amount of psilocybin, or a salt or solvate thereof, is 0.25 mg/kg.
Embodiment 18 provides the method of any one of embodiments 14-17, wherein the therapeutically effective amount is administered in a single dose to the subject.
Embodiment 19 provides the method of any one of embodiments 14-18, wherein the reduction, reversal, and/or elimination of OCD symptoms in the subject persists for at least 8 weeks after administration.
Embodiment 20 provides the method of any one of embodiments 14-19, wherein the subject was not treated with a contraindicated medication for at least 4 weeks before starting treatment with the 5-HT receptor agonist.
Embodiment 21 provides the method of any one of embodiments 14-20, wherein the reduction, reversal, and/or elimination of OCD symptoms in the subject is measured by the Yale-Brown Obsessive Compulsive Scale (YBOCS) score.
Embodiment 22 provides the method of any one of embodiments 14-21, wherein the subject has a pre-treatment YBOCS score of at least about 19.
Embodiment 23 provides the method of any one of embodiments 14-22, wherein one week after administering the 5-HT receptor agonist, the subject has a YBOCS score that is at least about 25% smaller than the pre-administration YBOCS score.
Embodiment 24 provides the method of any one of embodiments 14-23, wherein the reduced YBOCS persists for at least about 8 weeks after administration.
Embodiment 25 provides the method of any one of embodiments 14-24, wherein the method further includes the step of reducing, reversing, or eliminating anxiety.
Embodiment 26 provides the method of any one of embodiments 14-25, further comprising the step of administering a maintenance dose of the 5-HT receptor agonist upon symptom recurrence.
Embodiment 27 provides the method of any one of embodiments 14-26, wherein the obsessive thoughts are chosen from the group consisting of repeated thoughts about contamination, fear of losing control, repeated doubts, a need to have things in a particular order, superstitions, aggressive or horrific impulses, and sexual or violent imagery.
Embodiment 28 provides the method of any one of embodiments 14-27, wherein the compulsive behaviors are chosen from the group consisting of repetitive acts and mental acts.

What is claimed is:

1. A method of treating, preventing, or ameliorating obsessive-compulsive disorder (OCD) in a subject, the method comprising:
administering a therapeutically effective amount of a composition comprising a 5-HT receptor agonist to the subject, wherein the subject:
i) is suffering from OCD;
ii) is not diagnosed with obsessive-compulsive personality disorder (OCPD); and
reducing, reversing, or eliminating OCD symptoms in the subject.

2. The method of claim 1, wherein the 5-HT receptor agonist is psilocybin, or a salt, solvate, analog, or functional equivalent thereof.

3. The method of claim 2, wherein the effective amount of psilocybin, or a salt or solvate thereof, is a dose of about 0.05 to about 0.55 mg/kg.

4. The method of claim 3, wherein the effective amount of psilocybin, or a salt or solvate thereof, is 0.25 mg/kg.

5. The method of claim 1, wherein the therapeutically effective amount is administered in a single dose to the subject.

6. The method of claim 5, wherein the reduction, reversal, or elimination of OCD symptoms in the subject persists for at least 8 weeks after administration.

7. The method of claim 1, wherein the subject was not treated with a contraindicated medication for at least 4 weeks before starting treatment with the 5-HT receptor agonist.

8. The method of claim 1, wherein the reduction, reversal, and/or elimination of OCD symptoms in the subject is measured by the Yale-Brown Obsessive Compulsive Scale (YBOCS) score.

9. The method of claim 8, wherein the subject has a pre-treatment YBOCS score of at least about 19.

10. The method of claim 8, wherein one week after the administration of the 5-HT receptor agonist the subject has a YBOCS score that is at least about 25% smaller than the pre-administration YBOCS score.

11. The method of claim 10, wherein the reduced YBOCS persists for at least about 8 weeks after administration.

12. The method of claim 1, wherein the method further includes the step of reducing, reversing, or eliminating anxiety.

13. The method of claim 1, wherein the method further includes the step of administering a maintenance dose of the 5-HT receptor agonist upon symptom recurrence.

14. A method of treating, preventing, or ameliorating obsessive-compulsive disorder (OCD) in a subject, the method comprising:
administering a therapeutically effective amount of a composition comprising a 5-HT receptor agonist to the subject, wherein the subject:
i) is suffering from OCD;
ii) is not diagnosed with obsessive-compulsive personality disorder (OCPD); and
reducing, reversing, or eliminating obsessive thoughts or compulsive behaviors in the subject.

15. The method of claim 14, wherein the 5-HT receptor agonist is psilocybin, or a salt, solvate, analog, or functional equivalent thereof.

16. The method of claim 14, wherein the effective amount of psilocybin, or a salt or solvate thereof, is a dose of about 0.05 to about 0.55 mg/kg.

17. The method of claim 16, wherein the effective amount of psilocybin, or a salt or solvate thereof, is 0.25 mg/kg.

18. The method of claim 14, wherein the therapeutically effective amount is administered in a single dose to the subject.

19. The method of claim 14, wherein the reduction, reversal, or elimination of OCD symptoms in the subject persists for at least 8 weeks after administration.

20. The method of claim 14, wherein the subject was not treated with a contraindicated medication for at least 4 weeks before starting treatment with the 5-HT receptor agonist.

21. The method of claim 14, wherein the reduction, reversal, or elimination of OCD symptoms in the subject is measured by the Yale-Brown Obsessive Compulsive Scale (YBOCS) score.

22. The method of claim 21, wherein the subject has a pre-treatment YBOCS score of at least about 19.

23. The method of claim 21, wherein one week after administering the 5-HT receptor agonist, the subject has a YBOCS score that is at least about 25% smaller than the pre-administration YBOCS score.

24. The method of claim 21, wherein the reduced YBOCS persists for at least about 8 weeks after administration.

25. The method of claim 14, wherein the method further includes the step of reducing, reversing, or eliminating anxiety.

26. The method of claim 14, wherein the method further includes the step of administering a maintenance dose of the 5-HT receptor agonist upon symptom recurrence.

27. The method of claim 14, wherein the obsessive thoughts are chosen from the group consisting of repeated thoughts about contamination, fear of losing control, repeated doubts, a need to have things in a particular order, superstitions, aggressive or horrific impulses, and sexual or violent imagery.

28. The method of claim 14, wherein the compulsive behaviors are chosen from the group consisting of repetitive acts and mental acts.
